(19) United States
(12) Patent Application Publication
(10) Pub. No.: US 2024/0279745 A1
(43) Pub. Date: Aug. 22, 2024

(54) SYSTEMS AND METHODS FOR MULTI-ANALYTE DETECTION OF CANCER

(71) Applicant: Predicine, Inc., Hayward, CA (US)

(72) Inventors: Pan DU, Dublin, CA (US); Binggang XIANG, Pleasanton, CA (US); Chao DAI, Newark, CA (US); Shujun LUO, Castro Valley, CA (US); Shidong JIA, Palo Alto, CA (US)

(21) Appl. No.: 18/477,284

(22) Filed: Sep. 28, 2023

Related U.S. Application Data
(63) Continuation of application No. PCT/US2022/022664, filed on Mar. 30, 2022.
(60) Provisional application No. 63/168,436, filed on Mar. 31, 2021.

(51) Int. Cl. C12Q 1/6886; C12Q 1/6874

(52) U.S. Cl. CPC C12Q 1/6886; C12Q 1/6874; C12Q 2600/118; C12Q 2600/154; C12Q 2600/156

(57) ABSTRACT

Provided herein are methods and systems for multi-analyte detection of cancer. The methods may comprise assaying multiple nucleic acids to detect a set of biomarkers from samples. The methods may comprise processing the set of biomarkers to determine the presence of a cancer or cancer parameters. The processing may be performed by an algorithm. The algorithm may be a trained algorithm and may be trained on multiple training samples.

### CROSS-REFERENCE

This application is a continuation of International Application No. PCT/US2022/022664, filed Mar. 30, 2022, which claims the benefit of U.S. Provisional Patent Application No. 63/168,436, filed Mar. 31, 2021, each of which is incorporated by reference herein in its entirety.

### BACKGROUND

Cancer is a leading cause of deaths worldwide. Detection of cancer in individuals may be critical for providing treatment and improving patient outcomes. Cancer may be caused by genetic aberration which may lead to unregulated growth of calls. Detection of the genetic aberrations may be important for the detection of cancer. Sequencing of nucleic acids in a sample from a patient may be used to detect genetic aberrations.

### SUMMARY

Provided herein are systems and methods for detection of the presence or absence of cancer in a subject. The systems and methods provided herein comprises assaying polynucleotides to identify biomarkers of cancers in a subject. Detection of a type of cancer or the specific biomarkers for a given cancer may allow an effective treatment to be provided to an individual and may result in improved outcomes. For multiple types of cancer, the particular biomarkers that indicate a particular cancer type (or subtype) may be used to identify a prognosis for an individual suffering from the cancer. In order to provide accurate detection and prognosis for a cancer, multiple analytes may be examined. By analyzing an increased number of analytes (and sets of biomarkers from the analytes), the detection of a cancer (or cancer parameter) may be improved and may allow for the recommendation of an effective treatment, and may also allow for the prognosis to be more accurate.

In an aspect, the present disclosure provides a method for detecting a presence or an absence of cancer in a subject, comprising: (a) assaying cell-free deoxyribonucleic acid (cfDNA) molecules and cell-free ribonucleic (cfRNA) molecules from a biological sample obtained or derived from said subject to detect a first set of biomarkers from said cfDNA molecules and a second set of biomarkers from said cfRNA molecules; and (b) computer processing said first set of biomarkers and said second set of biomarkers to detect said presence or said absence of said cancer in said subject.

In some embodiments, the biological sample is selected from the group consisting of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a saliva sample, tissue biopsy, pleural fluid sample, peritoneal fluid sample, amniotic fluid sample, cerebrospinal fluid sample, lymphatic fluid sample, sweat sample, tear sample, semen sample, or any derivative thereof, and any combination thereof. In some embodiments, the biological sample comprises said plasma sample. In some embodiments, the biological sample comprises said urine sample.

In some embodiments, the cfDNA molecules and said cfRNA molecules are obtained or derived from a single biological sample of said subject. In some embodiments, the cfDNA molecules and said cfRNA molecules are obtained or derived from different biological samples of said subject.

In some embodiments, the biological sample is obtained or derived from said subject using an ethylenediaminetetraacetic acid (EDTA) collection tube, a cell-free RNA collection tube, or a cell-free deoxyribonucleic acid (DNA) collection tube, other blood collection tube, and CTC collection tubes.

In some embodiments, (a) comprises subjecting said biological sample to conditions that are sufficient to isolate, enrich, or extract said cfDNA molecules and said set of cfRNA molecules.

In some embodiments, the method further comprises fractionating a whole blood sample of said subject to obtain said cfDNA molecules and said cfRNA molecules.

In some embodiments, at least one of said cfDNA molecules and said cfRNA molecules are assayed using nucleic acid sequencing to produce nucleic acid sequencing reads. In some embodiments, the cfDNA molecules are assayed using DNA sequencing. In some embodiments, the DNA sequencing is selected from the group consisting of: next-generation sequencing, whole genome sequencing, low-pass sequencing, targeted sequencing, methylation-aware sequencing, enzymatic methylation sequencing, bisulfite methylation sequencing, and a combination thereof. In some embodiments, the DNA sequencing comprises low-pass whole genome sequencing. In some embodiments, the DNA sequencing comprises whole exome sequencing. In some embodiments, the DNA sequencing comprises methylation aware sequencing, enzymatic methylation sequencing or bisulfite methylation sequencing.

In some embodiments, the cfRNA molecules are assayed using RNA sequencing. In some embodiments, the RNA sequencing is selected from the group consisting of: next-generation sequencing, transcriptome sequencing, mRNA-seq, totalRNA-seq, smallRNA-seq, exosome sequencing, and a combination thereof. In some embodiments, the RNA sequencing comprises reverse transcribing said cfRNA molecules into complementary DNA (cDNA) molecules, and performing DNA sequencing on said cDNA molecules.

In some embodiments, the nucleic acid sequencing comprises nucleic acid amplification. In some embodiments, the nucleic acid amplification comprises polymerase chain reaction (PCR) or isothermal amplification. In some embodiments, the nucleic acid sequencing comprises use of substantially simultaneous reverse transcription (RT) and polymerase chain reaction (PCR).

In some embodiments, at least one of said cfDNA molecules and said cfRNA molecules are assayed using a polymerase chain reaction (PCR) assay, microarray, or a isothermal amplification.

In some embodiments, the cancer is selected from the group consisting of: breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof. In some embodiments, the cancer comprises said prostate cancer. In some embodiments, the prostate cancer is selected from the group consisting of: hormone sensitive prostate cancer (HSPC), castrate-resistant prostate cancer (CRPC), metastatic prostate cancer, and a combination thereof. In some embodiments, the subject is asymptomatic for said cancer. In some embodiments, the cancer comprises said breast cancer. In some embodiments, the cancer comprises bladder cancer.

In some embodiments, (b) comprises processing said first set of biomarkers and said second set of biomarkers using a trained algorithm. In some embodiments, the trained algorithm is trained using at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 independent training samples associated with a presence or an absence of said cancer. In some embodiments, the trained algorithm is trained using at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 independent training samples associated with a relapse of cancer. In some embodiments, the trained algorithm is trained using at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 independent training samples associated with a drug treatment or resistance to said drug treatment.

In some embodiments, the trained algorithm is trained using a first set of independent training samples associated with a presence of said cancer and a second set of independent training samples associated with an absence of said cancer. In some embodiments, the trained algorithm is trained using a first set of independent training samples associated with a presence of said cancer and a second set of independent training samples associated with a relapse of cancer. In some embodiments, the trained algorithm is trained using a first set of independent training samples associated with a presence of said cancer and a second set of independent training samples associated with a drug treatment or resistance to said drug treatment.

In some embodiments, the method further comprises using said trained algorithm or another trained algorithm to process a set of clinical health data of said subject to determine said presence or said absence of said cancer. In some embodiments, the method further comprises using said trained algorithm or another trained algorithm to process a set of clinical health data of said subject to determine a relapse of cancer. In some embodiments, the method further comprises using said trained algorithm or another trained algorithm to process a set of clinical health data of said subject to determine a drug treatment or resistance to said drug treatment.

In some embodiments, the trained algorithm comprises an un-supervised machine learning algorithm. In some embodiments, the trained algorithm comprises a supervised machine learning algorithm. In some embodiments, the supervised machine learning algorithm comprises a deep learning algorithm, a support vector machine (SVM), a neural network, or a Random Forest.

In some embodiments, (b) comprises detecting said presence or said absence of said cancer in said subject at an accuracy of at least about 60%, at least about 70%, at least about 75%, at least about 80%, at least about 85%, at least about 90%, at least about 95%, at least about 98%, or at least about 99%.

In some embodiments, (b) comprises detecting said presence or said absence of said cancer in said subject at a sensitivity of at least about 60%, at least about 70%, at least about 75%, at least about 80%, at least about 85%, at least about 90%, at least about 95%, at least about 98%, or at least about 99%.

In some embodiments, (b) comprises detecting said presence or said absence of said cancer in said subject at a specificity of at least about 60%, at least about 70%, at least about 75%, at least about 80%, at least about 85%, at least about 90%, at least about 95%, at least about 98%, or at least about 99%.

In some embodiments, (b) comprises detecting said presence or said absence of said cancer in said subject at a positive predictive value of at least about 60%, at least about 70%, at least about 75%, at least about 80%, at least about 85%, at least about 90%, at least about 95%, at least about 98%, or at least about 99%.

In some embodiments, (b) comprises detecting said presence or said absence of said cancer in said subject at a negative predictive value of at least about 60%, at least about 70%, at least about 75%, at least about 80%, at least about 85%, at least about 90%, at least about 95%, at least about 98%, or at least about 99%.

In some embodiments, said biological sample is obtained or derived from said subject prior to said subject receiving a therapy for said cancer. In some embodiments, said biological sample is obtained or derived from said subject during a therapy for said cancer. In some embodiments, said biological sample is obtained or derived from said subject after receiving a therapy for said cancer.

In some embodiments, said therapy is selected from the group consisting of: surgical resection, chemotherapy, radiotherapy, immunotherapy, cell therapy, adjuvant therapy, neoadjuvant therapy, androgen deprivation therapy, and a combination thereof.

In some embodiments, the method further comprises identifying a clinical intervention for said subject based at least in part on said detected presence or said absence of said cancer. In some embodiments, said clinical intervention is selected from a plurality of clinical interventions. In some embodiments, said clinical intervention is selected from the group consisting of: surgical resection, chemotherapy, radiotherapy, immunotherapy, adjuvant therapy, neoadjuvant therapy, androgen deprivation therapy, and a combination thereof. In some embodiments, the method further comprises administering said clinical intervention to said subject.

In some embodiments, said first set of biomarkers comprises quantitative measures of a first set of cancer-associated genomic loci. In some embodiments, said first set of cancer-associated genomic loci comprises one or more members selected from the group consisting of genes listed in Table 1. In some embodiments, said first set of cancer-associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 1. In some embodiments, the first set of cancer-associated genomic loci comprises PTEN, TP53 or RB1. In some embodiments, the first set of cancer-associated genomic loci comprises PTEN, TP53 and RB1. In some embodiments, the first set of cancer-associated genomic loci comprises PTEN. In some embodiments, the first set of cancer-associated genomic loci comprises FGFR3 or ERBB2.

In some embodiments, said second set of biomarkers comprises quantitative measures of a second set of cancer-associated genomic loci. In some embodiments, said second set of cancer-associated genomic loci comprises one or more members selected from the group consisting of genes listed in Table 2. In some embodiments, said second set of cancer-associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 2.

In some embodiments, the method further comprises using probes configured to selectively enrich said biological sample for nucleic acid molecules corresponding to a set of genomic loci. In some embodiments, said probes are nucleic acid primers. In some embodiments, said probes have sequence complementarity with at least a portion of nucleic acid sequences of said set of genomic loci. In some embodiments, said probes comprise at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 different probes.

In some embodiments, the method further comprises determining a likelihood of said determination of said presence or said absence of said cancer in said subject.

In some embodiments, the method further comprises monitoring said presence or said absence of said cancer in said subject, wherein said monitoring comprises assessing said presence or said absence of said cancer in said subject at each of a plurality of time points.

In some embodiments, a difference in said assessment of said presence or said absence of said cancer in said subject among said plurality of time points is indicative of one or more clinical indications selected from the group consisting of: (i) a diagnosis of said cancer, (ii) a prognosis of said cancer, and (iii) an efficacy or non-efficacy of a course of treatment for treating said cancer of said subject. In some embodiments, said prognosis comprises an expected progression-free survival (PFS) or overall survival (OS).

In some embodiments, the method further comprises assaying germline DNA (gDNA) molecules obtained or derived from said subject to detect a third set of biomarkers, and computer processing said third set of biomarkers to detect said presence or said absence of said cancer in said subject.

In some embodiments, said first set of biomarkers from said cfDNA molecules comprise tumor-associated alterations selected from the group consisting of: copy number alterations (CNAs), copy number losses (CNLs), loss of heterozygosity (LOH), single nucleotide variants (SNVs), insertions or deletions (indels), rearrangements, and epigenetic changes such as methylation. In some embodiments, the first set of biomarkers from said cfDNA molecules comprise copy number variation. In some embodiments, the first set of biomarkers from said cfDNA molecules comprise copy number losses. In some embodiments, the first set of biomarkers from said cfDNA molecules comprise single nucleotide variants.

In some embodiments, said second set of biomarkers from said cfRNA molecules comprise tumor-associated alterations selected from the group consisting of: alternative splicing variants, fusions, single nucleotide variants (SNVs), and insertions or deletions (indels).

In some embodiments, the method further comprises filtering at least a subset of said nucleic acid sequencing reads based on a quality score.

In some embodiments, the method further comprises performing error correction on said nucleic acid sequencing reads using sample barcodes or molecular barcodes attached to at least one of said cfDNA molecules and said cfRNA molecules.

In some embodiments, the method further comprises performing at least one of single-stranded consensus calling and double-stranded consensus calling on said nucleic acid sequencing reads, thereby suppressing sequencing and PCR errors in said nucleic acid sequencing reads.

In some embodiments, the method further comprises determining, among said first set of biomarkers, a mutant allele frequency of a set of somatic mutations. In some embodiment, the method further comprises determining a blood copy number burden based on copy number alterations or copy number losses of said first set of biomarkers.

In some embodiments, the method further comprises determining a circulating tumor DNA (ctDNA) fraction of said cancer of said subject based at least in part on said set of mutant allele frequencies.

In some embodiments, the method further comprises determining a plasma tumor mutational burden (pTMB) of said cancer of said subject based at least in part on said set of mutant allele frequencies.

In some embodiments, the method further comprises determining a plasma tumor mutational burden (pTMB) of said cancer of said subject based at least in part on said set of mutant allele frequencies comprising microsatellites.

In some embodiments, the method further comprises determining an abnormality score of said cancer of said subject based at least in part on said set of mutant allele frequencies.

In some embodiments, the method further comprises determining a methylation related score of said cancer of said subject based at least in part on said set of mutant allele frequencies.

In another aspect, the present disclosure provides a method for detecting a presence or an absence of prostate cancer in a subject, comprising: (a) assaying cell-free deoxyribonucleic acid (cfDNA) molecules and germline DNA (gDNA) molecules from a biological sample obtained or derived from said subject to detect a first set of biomarkers from said cfDNA molecules and a second set of biomarkers from said gRNA molecules, wherein at least one of said first set of biomarkers and said second set of biomarkers comprises an androgen receptor (AR) alteration; and (b) computer processing said first set of biomarkers and said second set of biomarkers to detect said presence or said absence of said prostate cancer in said subject.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1A-1C shows plots of distributions of ctDNA fractions, pTMB (plasma tumor burden), and ctDNA across metastatic groups.

FIG. 2 shows plots of Distribution of cfDNA yields based on metastatic volume in the untreated hormone-sensitive group and serum alkaline phosphatase (ALP) in mCRPC states.

FIG. 3A shows plots of Combined analysis of ctDNA fraction and metastatic volume for the prediction of ADT failure in mHSPC patients. FIG. 3B shows plots Overall survival in the mHSPC group based on the combined analysis of volume of metastatic disease with ctDNA fraction in mHSPC patients. FIG. 3C shows plots of Combined analysis of ctDNA fraction and serum ALP levels of overall survival in mCRPC patients.

FIG. 4A shows plots of individual patient ctDNA fractions and variant counts across metastatic groups. FIG. 4B shows overall heatmap of individual somatic alterations observed in metastatic prostate cancer groups. FIG. 4C shows plots of overall heatmap of deleterious/likely deleterious alterations detected in genes involved in DNA damage repair pathways.

FIGS. 5A and 5B show alteration frequencies in key genes between mCRPC and mHSPC groups. FIG. 5C shows a lollipop plot of AR somatic mutations detected in mHSPC and mCRPC patients. FIG. 5D shows a distribution of AR hotspot mutations across exon regions in mCRPC patients. FIG. 5E shows distribution of AR mutations and AR copy number gain along with matching ctDNA fractions in mCRPC patients detected with these alterations.

FIG. 6A shows PSA changes after 3-months of ADT in untreated mHSPC paired patient samples. FIG. 6B shows ctDNA fraction changes after 3-months of ADT in untreated mHSPC paired patient samples. FIG. 6C shows ctDNA-based somatic alterations of top frequently mutated genes detected in 29 paired untreated mHSPC patients before and after 3 months of androgen deprivation therapy.

FIG. 7A shows plots of RB1 wild type vs copy number deletion and overall survival in mCRPC patients. FIG. 7B shows AR copy number gain compared to wild type and overall survival in mCRPC patients. FIG. 7C shows TP53 mutations vs wild type and overall survival in mCRPC patients.

FIG. 8 shows plots relating to the correlation of MSI status with plasma TMB.

FIG. 9A shows Overall survival in untreated mHSPC group based on detectable genomic events. FIG. 9B shows ADT failure in untreated mHSPC group based on detectable genomic events.

FIG. 9C shows Overall survival in combined mCRPC groups based on detectable genomic events

FIG. 10 shows plot of landscape of AR aberrations identified in cell-free DNA and RNA.

FIGS. 11A-11I show Kaplan-Meier analysis of PSA-PFS, clinical or radiographic PFS, and overall survival, according to AR copy number status, the presence of at least one of AR gain, AR splice variant, or AR somatic mutation, and the total number of AR aberrations (0, 1,2) present. FIG. 11J shows univariable Cox proportional hazard analysis of clinical endpoints based on AR aberrations in two independent cohorts. FIG. 11K shows Multivariable Cox proportional hazard analysis of clinical endpoints based on AR aberrations

FIG. 12A shows Kaplan-Meier analysis of PSA-PFS, according to concurrent expression of both an AR-V and an AR copy number gain. FIG. 12B shows Kaplan-Meier analysis of clinical or radiographic PFS, according to concurrent expression of both an AR-V and an AR copy number gain. FIG. 12C shows Kaplan-Meier analysis overall survival, according to concurrent expression of both an AR-V and an AR copy number gain.

FIG. 13 shows Cox proportional hazards analysis of clinical outcomes based on PI3K/AR pathway aberrations.

FIGS. 14A-14B show example schematics of workflows for methods disclosed.

FIGS. 15A-15C show data for paired tumor tissue-plasma samples of metastatic castration-resistant prostate cancer patients.

FIGS. 16A-16E show analysis of 52 mCRPC plasma samples relating to genomic alterations of TP53, RB1, and PTEN as well as overall survival (OS).

FIG. 17 shows correlation between copy numbers estimated from liquid and tissue biopsies for genes with both tissue and liquid biopsy CNV calls in the paired samples.

FIGS. 18A-18D show the patient cohort and study design for detection of tumor suppressor gene copy number loss.

FIGS. 19A-19F show charts relating to somatic aberrations detected in tumor tissues and liquid biopsies

FIGS. 20A-20F show the clinical implications of utDNA analysis in urothelial bladder cancer.

FIGS. 21A-21G show charts relating bTMB and patient outcomes.

FIGS. 22A-22D demonstrate dynamic changes in bCNB predict patient outcomes and precede radiographic response and clinical progression.

FIG. 23 shows a schematic of the general workflow of the plasma WES and methylation profiling.

FIGS. 24A-24E show the NGS technical performance comparison of Predicine ECM vs WGBS (whole genome bisulfite sequencing)

FIG. 25 shows the differentially methylated region (DMR) analysis of cancer and normal samples.

FIG. 26 shows a computer system that is programmed or otherwise configured to implement methods provided herein.

FIG. 27 shows variant frequency for androgen receptors detected in cfRNA and cfDNA.

FIG. 28 shows detection of fusion events using ctRNA.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are systems and methods for detection of the presence or absence of cancer in a subject. The systems and methods provided herein comprises assaying polynucleotides to identify biomarkers of cancers in a subject. The biomarkers may be processed in order to identify the presence or absence of cancer. The methods described herein may process multiple type of analytes in order to determine a presence or absence of cancer. The multiple types of analytes may comprise DNA or RNA, for example cfDNA or cfRNA. The multiple analytes may be cfDNA, germline DNA, and cfRNA. By analyzing a plurality of different analytes the methods may allow for improved detection or determination of a prognosis as compared to methods performed on fewer analytes or only one of many different analytes.

In an aspect, the present disclosure provides a method for detecting a presence or an absence of cancer in a subject, comprising: (a)assaying cell-free deoxyribonucleic acid (cfDNA) molecules and cell-free ribonucleic (cfRNA) molecules from a biological sample obtained or derived from said subject to detect a first set of biomarkers from said cfDNA molecules and a second set of biomarkers from said cfRNA molecules; and (b) computer processing said first set of biomarkers and said second set of biomarkers to detect said presence or said absence of said cancer in said subject.

The subject may be a suspected of a suffering from a cancer. The cancer may be specific or originating from an organ or other area of the subject. For example, the cancer may be breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof. The cancer may be a hormone sensitive prostate cancer (HSPC), castrate-resistant prostate cancer (CRPC), metastatic prostate cancer, and a combination thereof. The cancer may comprise biomarkers that are specific to a particular cancer. The specific biomarkers may indicate a presence of a particular cancer. For example, biomarker may indicate that a castrate-resistant prostate cancer is present. The identification of the presence of a type of cancer may allow the determination of a treatment option or recommendation.

In some cases, the subject may be asymptomatic for cancer. For example, the cancer may not exhibit any symptoms and the subject may be unaware of the presence of cancer. The methods described herein may allow a cancer to be identified at an earlier stage than otherwise. The identification of the presence of the cancer at an earlier stage may allow a treatment option or recommendation to be determined at an earlier stage and may allow the subject to have an improved prognosis.

The biological sample may comprise nucleic acids. The biological sample be a cell-free deoxyribonucleic acid (cfDNA) sample or a cell-free ribonucleic acid (cfRNA) sample. The biological sample may comprise genomic DNA or germline DNA(gDNA). The nucleic acid may be a DNA (e.g. double-stranded DNA, single-stranded DNA, single-stranded DNA hairpins, cDNA, genomic DNA, germline DNA, circulating tumor DNA (ctDNA), cell-free DNA (cfDNA)), an RNA (e.g. cfRNA, mRNA, CRNA, miRNA, siRNA, miRNA, snoRNA, piRNA, tiRNA, snRNA), or a DNA/RNA hybrids. The biological sample may be a derived from or contain a biological fluid. For example, the biological sample may be a plasma sample, a serum sample, a buffy coat sample, a peripheral blood mononuclear cell (PBMC) sample, a red blood cell sample, a urine sample, a saliva sample, or other body fluid sample. The biological sample may comprise or be a pleural fluid sample, peritoneal fluid sample, amniotic fluid sample, cerebrospinal fluid sample, lymphatic fluid sample, sweat sample, tear sample, semen sample, or any combination of biological fluid. In some case, the samples may comprise RNA and DNA. For example, a sample may comprise cfDNA and cfRNA and the cfDNA and cfRNA may be analyzed by methods as described elsewhere herein.

The biological sample may be collected, obtained, or derived from said subject using a collection tube. The collection tube may be an ethylenediaminetetraacetic acid (EDTA) collection tube, a cell-free RNA collection tube, or a cell-free deoxyribonucleic acid (DNA) collection tube and CTC collection tubes, or other blood collection tube. The collection tube may comprise additional reagents for stabilizing the nucleic acid molecules or blood cells. The collection tube may allow the nucleic acid or blood cells to be stable such to minimize degradation of the biological sample prior to assaying. The additional reagents may comprise buffer salts or chelators.

The biological sample may be obtained or derived from a subject at a various times. The biological sample may be obtained or derived from a subject prior to the subject receiving a therapy for cancer. The biological sample may be obtained or derived from a subject during receiving a therapy for cancer. The biological sample may be obtained or derived from a subject after receiving a therapy for cancer. The biological sample may be collected over 1, 2, 3, 4, 5, 6, 7, 8, 9, 10, 11, 12, 15, 20, 30, 40, 50, 60, 70, 80, 90, 100, 200, 300, 400, 500, 600, 700, 800, 900, 1000 or time points. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more hour period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more day period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more week period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more month period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, SS, 60 or more year period.

In various aspects as described herein, a clinical intervention or a therapy may be identified at least in part based on the identification of the presences of cancer, or the presence of a parameter of cancer. The clinical intervention may be a plurality of clinical interventions. The clinical intervention may be selected from a plurality of clinical interventions. The clinical intervention may be a surgical resection, chemotherapy, radiotherapy, immunotherapy, adjuvant therapy, neoadjuvant therapy, androgen deprivation therapy, or a combination thereof. In some cases, the clinical interventions may be administered to the subject. After administration of the clinical intervention, a sample may be obtained or derived from the subject such to monitor the cancer or cancer parameters. As such, the methods and systems disclosed herein may be performed iteratively such that monitoring of a cancer can be performed. Additionally, by performing the methods or systems iteratively, therapies or clinical interventions may be updated based on the results of the methods. The monitoring of the cancer may include an assessment as well as a difference in assessment from a previously generated assessment. The difference in an assessment of cancer in said subject among a plurality of time points (or samples) may be indicative of one or more clinical indications such as a diagnosis of said cancer, a prognosis of said cancer, or an efficacy or non-efficacy of a course of treatment for treating said cancer of said subject. The prognosis may comprise expected progression-free survival (PFS), overall survival (OS), or other metrics relating the severity or survivability of a cancer

The biological samples may be subjected to additional reactions or conditions prior to assaying. For example, the biological sample may be subjected to conditions that are sufficient to isolate, enrich, or extract nucleic acids, such cfDNA molecules or cfRNA molecules.

The methods disclosed herein may comprise conducting one or more enrichment reactions on one or more nucleic acid molecules in a sample. The enrichment reactions may comprise contacting a sample with one or more beads or bead sets. The enrichment reactions may comprise one or more hybridization reactions. For example, the enrichment reactions may comprise contacting a sample with one or more capture probes or bait molecules that hybridize to a nucleic acid molecule of the biological sample. The enrichment reaction may comprise differential amplification of a set of nucleic acid molecules. The enrichment reaction may enrich for a plurality of genetic loci or sequences corresponding to genetic loci. For example, the enrichment reaction may enrich for sequences corresponding to genes from Table 1 or Table 2. The enrichment reactions may comprise the use of primers or probes that may complementarity to sequences (or sequences upstream or downstream) of a sequence that is to be enriched. For example, a capture probe may comprise sequence complementarity to a set of genomic loci and allow the enrichment of the genomic loci. The enrichments reactions may comprise a plurality of probes or primers. A plurality of probes may comprise 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 different probes.

The methods disclosed herein may comprise conducting one or more isolation or purification reactions on one or more nucleic acid molecules in a sample. The isolation or purification reactions may comprise contacting a sample with one or more beads or bead sets. The isolation or purification reaction may comprise one or more hybridization reactions, enrichment reactions, amplification reactions, sequencing reactions, or a combination thereof. The isolation or purification reaction may comprise the use of one or more separators. The one or more separators may comprise a magnetic separator. The isolation or purification reaction may comprise separating bead bound nucleic acid molecules from bead free nucleic acid molecules. The isolation or purification reaction may comprise separating capture probe hybridized nucleic acid molecules from capture probe free nucleic acid molecules. The isolation reactions may comprises removing or separating a group of nucleic acid molecules from another group of nucleic acids.

The methods disclosed herein may comprise conduction extraction reactions on one or more nucleic acids in a biological sample. The extraction reactions may lyse cells or disrupt nucleic acid interactions with the cell such that the nucleic acids may be isolated, purified, enriched or subjected to other reactions.

The methods disclosed herein may comprise amplification or extension reactions. The amplification reactions may comprise polymerase chain reaction. The amplification reaction may comprise PCR-based amplifications, non-PCR based amplifications, or a combination thereof. The one or more PCR-based amplifications may comprise PCR, qPCR, nested PCR, linear amplification, or a combination thereof. The one or more non-PCR based amplifications may comprise multiple displacement amplification (MDA), transcription-mediated amplification (TMA), nucleic acid sequence-based amplification (NASBA), strand displacement amplification (SDA), real-time SDA, rolling circle amplification, circle-to-circle amplification or a combination thereof. The amplification reactions may comprise an isothermal amplification.

The method disclosed herein may comprise a barcoding reaction. A barcoding reaction may comprise the additional of a barcode or tag to the nucleic acid. The barcode may be a molecular barcode or a sample barcode. For example, a barcode nucleic acid may comprise a barcode sequence which may be a degenerate n-mer. The sequence may be randomly generated or generated such to synthesize a specific barcode sequence. The barcode nucleic acid may be added to a sample such to label the nucleic acid molecules in the sample. The barcodes may be specific to a sample. For example, a plurality of barcode nucleic acids may be added to a sample in which the barcode sequence is the same. Upon barcoding of the nucleic acids, those originating from a same sample may have a same barcode sequence, and may allow a nucleic acid to be identified as belonging to a particular or given sample. A molecular barcode may also be used such that each molecule (or a plurality of molecules) in a same volume have a different molecular barcode. This barcode may be subjected to amplification such that all amplicons derived from a molecule have the same barcode. In this way, molecules originating from a same molecule may be identified. The sequences reads may be processed based on the barcode sequences. For example, the processing may reduce errors or allow a molecule to be tracked. Barcode sequences may be appended or otherwise added or incorporated into a sequence by various reactions, for example an amplification, extension, or ligation reaction, and may be performed enzymatically using a nucleic acid polymerase or ligase. The ligation may be an overhang or blunt end ligation and the barcodes may comprise complementarity to nucleic acids to be barcoded. This complementarity may be a sequence derived from the sample from the subject or may be constant sequence generated via a reaction performed on the nucleic acids in the sample.

In some cases, the biological sample may comprise multiple components. For example, the biological sample may be a whole blood sample. The biological sample may be subjected to reactions such to separate or fractionate a biological sample. For example, a whole blood sample may be a fractionated and cell free nucleic acids may be obtained. The whole blood sample may be fractionated using centrifugation such that blood cells may be separated from the plasma (which may contain cell free nucleic acid). A sample may be subjected to multiple rounds of separation or fractionation.

In various aspects described throughout the disclosure, the nucleic acids may be subjected to sequencing reactions. The sequencing the reactions may be used on DNA, RNA or other nucleic acid molecules. Example of a sequencing reaction that may be used include capillary sequencing, next generation sequencing, Sanger sequencing, sequencing by synthesis, single molecule nanopore sequencing, sequencing by ligation, sequencing by hybridization, sequencing by nanopore current restriction, or a combination thereof. Sequencing by synthesis may comprise reversible terminator sequencing, processive single molecule sequencing, sequential nucleotide flow sequencing, or a combination thereof. Sequential nucleotide flow sequencing may comprise pyrosequencing, pH-mediated sequencing, semiconductor sequencing or a combination thereof. The sequencing reactions may comprise whole genome sequencing, whole exome sequencing, low-pass whole genome sequencing, targeted sequencing, methylation-aware sequencing, enzymatic methylation sequencing, bisulfite methylation sequencing. The sequencing reaction may be a transcriptome sequencing, mRNA-seq, totalRNA-seq, smallRNA-seq, exosome sequencing, or combinations thereof. Combinations of sequencing reactions may be used in the methods described elsewhere herein. For example, a sample may be subjected to whole genome sequencing and whole transcriptome sequencing. As the samples may comprise multiple types of nucleic acids (e.g. RNA and DNA), sequencing reactions specific to DNA or RNA may be used such to obtain sequence reads relating to the nucleic acid type.

The sequencing of nucleic acids may generate sequencing read data. The sequencing reads may be processed such to generate data of improved quality. The sequencing reads may be generated with a quality score. The quality score may indicate an accuracy of a sequence read or a level or signal above a nose threshold for a given base call. The quality scores may be used for filtering sequencing reads. For example, sequencing reads may be removed that do not meet a particular quality score threshold. The sequencing reads may be processed such to generate a consensus sequence or consensus base call. A given nucleic acid (or nucleic acid fragment) may be sequenced and errors in the sequence may be generated due to reactions prior or during sequencing. For example, amplification or PCR may generate error in amplicons such that the sequences are not identical to a parent sequence. Using sample barcodes or molecular barcodes, error correction may be performed. Error correction may include identifying sequence reads that do not corroborate with other sequences from a same sample or same original parent molecules. The use of barcodes may allow the identification or a same parent or sample. Additionally, the sequence reads may be processed by performing single strand consensus calling or double stranded consensus call, thereby reducing or suppressing error.

The methods as disclosed herein may comprise determining allele frequency or other cancer related metric. The methods may comprise a mutant allele frequency of a set of somatic mutation among a set of biomarkers. The mutant allele frequency may be used to determine a circulating tumor DNA (ctDNA) fraction of a cancer of a subject. A plasma tumor mutational burden (pTMB) of a cancer of the subject may be determined based at least in part on the set of mutant allele frequencies. Detection of microsatellite instability may also be used to determine the presence or absence of a cancer or cancer metric. Methylation states may be determined using methods described herein and may be used to identify a presence of a cancer or cancer parameter.

In various aspects, sets of biomarkers are processed and data corresponding to the biomarkers are generated. The sets of biomarkers may comprise quantitative measures from a set of cancer-associated genomic loci. The cancer-associated genomic loci may correspond to a set of genes. The cancer associated genomic loci may comprise one or more genes selected from Table 1. In some case, a set of cancer associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 1. The cancer associated genomic loci may comprise one or more genes selected from Table 2. In some case, a set of cancer associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 2.

TABLE 1
List of genes

| | | | | | | | |
|---|---|---|---|---|---|---|---|
| ABRAXAS1 | AKT1 | ALK | APC | AR | ARAF | ARIDIA | ATM |
| ATR | BAP1 | BARD1 | BRAF | BRCA1 | BRCA2 | BRIP1 | CCND1 |
| CCNE1 | CD274 | CDH1 | CDK12 | CDK4 | CDK6 | CDKN2A | CHEK1 |
| CHEK2 | CTNNB1 | DDR2 | DNAJB1 | EGFR | EPCAM | ERBB2 | ERBB3 |
| ERCC1 | ERCC2 | ERCC4 | ESR1 | EZH2 | FANCA | FANCC | FANCD2 |
| FANCI | FANCL | FANCM | FBXW7 | FGFR1 | FGFR2 | FGFR3 | GNA11 |
| GNAQ | GNAS | HDAC2 | HRAS | IDH1 | IDH2 | JAK2 | JAK3 |
| KIT | KRAS | MAP2K1 | MAP2K2 | MAPK1 | MDM2 | MET | MLH1 |
| MPL | MRE11 | MSH2 | MSH6 | MTOR | MYC | MYCN | MYD88 |
| NBN | NF1 | NFE2L2 | NPM1 | NRAS | NTRK1 | NTRK3 | PALB2 |
| PDCDILG2 | PDGFRA | PIK3CA | PMS2 | POLD1 | POLE | PPM1D | PPP2R1A |
| PPP2R2A | PRKACA | PRKD1 | PTEN | PTPN11 | RAD50 | RAD51 | RAD51B |
| RAD51C | RAD51D | RAD54L | RAF1 | RB1 | RECQL | RET | RNF43 |
| ROS1 | RPA1 | SMAD4 | SMO | SPOP | STK11 | TERT | TMPRSS2 |
| TP53 | TP53BP1 | TSC1 | TSC2 | VHL | XRCC2 | XRCC3 | XRCC4 |

TABLE 2
List of genes in PredicineCARE panel
Gene List
PredicinePLUS DNA + RNA Panel
SNV and Indel (158 key cancer related genes)

| | | | | | | | | | | |
|---|---|---|---|---|---|---|---|---|---|---|
| ABL1 | BRCA1* | CREBBP | EZH1 | H3F3A | MAP2K1 | NF1* | PIK3CA* | RADS1C* | SMARCA4 | VEGFA* |
| AKT1 | BRCA2* | CRKL* | EZH2 | H3F3B | MAP2K2 | NF2* | PIK3CB | RADS1D | SMARCB1* | VEGFB |
| ALK | BTK | CSF1R | FANCA | HNF1A | MAPK1 | NFE2L2 | PIK3CD | RAF1 | SMO* | VHL |
| APC* | CALR | CTNNB1 | FBXW7 | HRAS | MDM2* | NOTCH1 | PIK3R1* | RB1* | SPOP | WT1 |
| AR* | CBL | CXCR4 | FGFR1* | IDH1 | MED12 | NPM1 | PMS2* | RET | SR5F2 | ZNF148 |
| ARAF | CCND1* | DDR2 | FGFR2* | IDH2 | MET* | NRAS | POLD1 | RHEB | STAG2 | |
| ARIDIA* | CCND2 | DICER1 | FGFR3* | IKZF1 | MLH1* | NTRK1* | POLE | RHOA | STAT3 | |
| ARID2 | CCND3 | DNMT3A | FGFR4* | JAK2 | MPL | NTRK2* | PPP2R1A | RIT1 | STK11 | |
| ASXL1 | CD274* | EGFR* | FLT3 | JAK3 | MSH2* | NTRK3* | PRKACA | RNF43 | TERT | |
| ATM* | CD79B | EPCAM | FOXL2* | KCNI5 | MSH6* | PALB2* | PRKD1 | ROS1 | TET2 | |
| ATR | CDH1* | ERBB2* | GATA2 | KDM6A* | MTOR | PAX5* | PTCH1* | RUNX1 | TGFBR2 | |
| ATRX | CDK4* | ERBB3 | GATA3 | KDR | MUTYH | PBMR1* | PTEN* | SERPINB3* | TPS3* | |
| B2M | CDKG* | ERBB4 | GNA11 | KIT* | MYC* | PDCD1LG2* | PTPN11* | SERPINB4* | TSC1 | |
| BAP1* | CDKN2A* | ERRFI1* | GNAQ | KMT2C | MYCN* | PDGFRA | RAC1 | SF3B1 | TSC2 | |
| BRAF | CHEK2* | ESR1 | GNAS | KRAS* | MYD88 | PDGFEB | RADS1B* | SMAD4 | U2AF1 | |

Chemotherapy response related genes (9 genes)

| | | | | | | | | |
|---|---|---|---|---|---|---|---|---|
| CYP2C19 | CYP2D6 | CYP3A4 | DPYD | GSTP1 | MTHFE | UGT1A1 | XPC | XRCC1 |

Copy number alteration detection only (15 genes)

| | | | | | | | | | | |
|---|---|---|---|---|---|---|---|---|---|---|
| AKT2 | AREG | AURKA | BCL2 | CCNE1 | CDKN2B | EREG | FGF19 | FGF3 | FGF4 | HGF |
| IGF1R | IGF | TOP2A | PPP2RA | | | | | | | |

Detection of Select Rearrangements by Breakpoints: 12 genes (11 genes present above)

| | | | | | | | | | | |
|---|---|---|---|---|---|---|---|---|---|---|
| ALK | FGFR1 | FGFR3 | MET | PDGFRA | RET | CD74 | EGFR | FGFR2 | MET | NTRK1 |
| PDGFEB | RDS1 | | | | | | | | | |

*Indicated genes covered by full coding regions

The sets of biomarkers may correspond to genetic aberration of a genetic locus. The genetic aberration may a tumor associated alteration. The genetic aberration may be a copy number alterations (CNAs), copy number losses (CNLs), single nucleotide variants (SNVs), insertions or deletions (indels), and rearrangements. The set of biomarkers may be identified in a variety of nucleic acid types. For example, the tumor associated alteration may be identified in cfDNA or cfRNA. The tumor associated alteration may comprise changes in allelic expression, or gene expression. Methods and systems disclosed herein may allow for gene expression profiling and identification of changes to the expression levels of gene.

In various aspects, the methods may comprise identifying the presence of a cancer or a cancer parameter. The methods may comprises determining a probability or a likelihood of the presence of cancer or a cancer parameter. For example, instead of a binary output indicating a presence or absence, an output may be generated that indicates a probability that subject has cancer. This probability may be determined based on algorithms as described elsewhere herein. Similarly, a probability or likely of response to a particular treatment or a probability of relapse may be outputted.

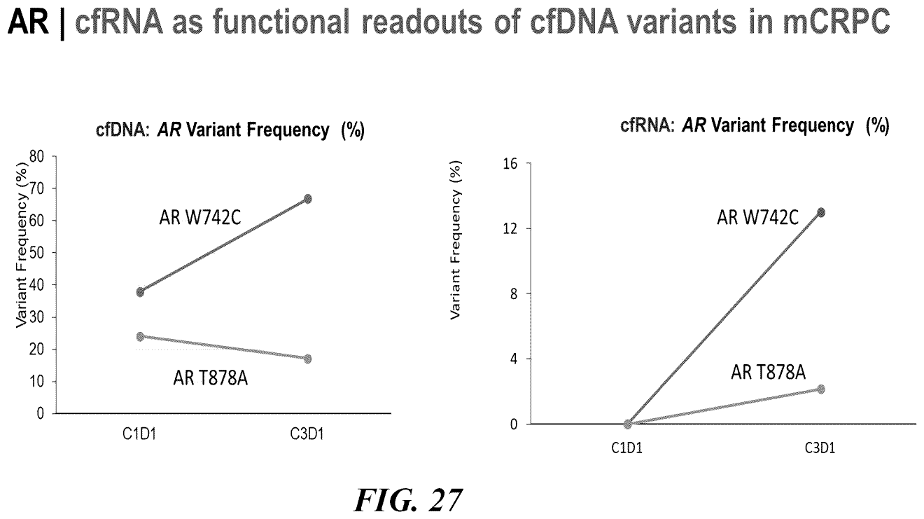

The increased cfRNA transcriptional expression of drug resistance-related gene alterations or splicing variants may serve as predictive biomarker, identifying the response or resistance to therapy. Specifically, in the case of prostate cancer, the increased cfRNA transcriptional expression of drug resistance-related AR mutations such as W742C/L and F877L or splicing variants such as AR-V7 or AR-V9, may serves as predictive biomarker, identifying the response or resistance to anti-androgen therapy (see FIG. 27)

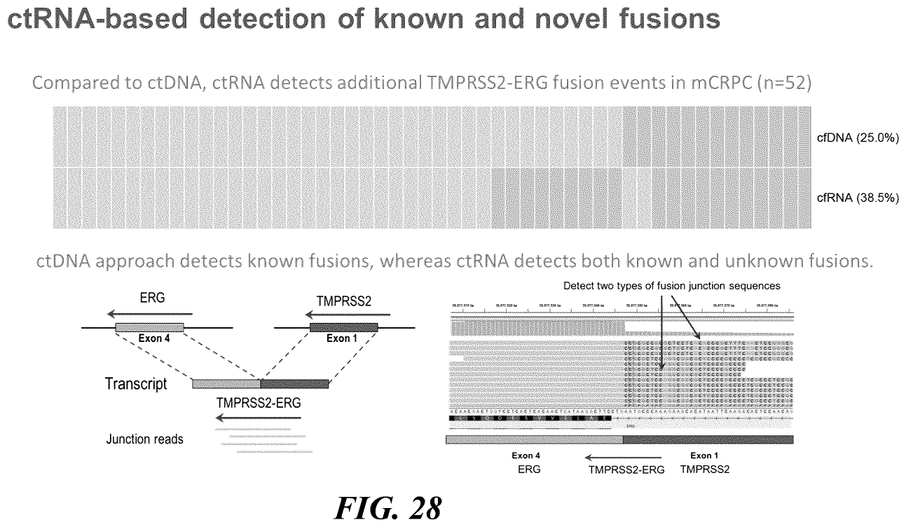

Compared to the use of cfDNA, blood ctRNA-based variant detection (including fusion) can be used to be more effectively to identify known and novel variants especially fusions in cancer. For instance, blood cfRNA based detection of TMPRSS2-ERG provides higher detection sensitivity in prostate cancer (see FIG. 28).

The increased ratio of blood-based cancer variants versus urine-based cancer variants could serve as a prognostic biomarker in GU cancers, indicating the disease aggressiveness and guide clinical treatment decision making. Specifically, in the case of muscle-invasive bladder cancer (MIBC), the increased level of blood-based cancer variants versus urine-based cancer variants could serve as a prognostic biomarker in patients with MIBC and provide evidence for clinical decision making. These cancer variants may include ctDNA, cfRNA, microRNA, methylation, among others.

Together with cfDNA based variant detection through genomics and epigenomics, cfRNA and/or microRNA can also be used either alone or in combination with genomic and epigenomic biomarkers for minimal residual disease (MRD) detection, therapy monitoring and early cancer detection.

In various aspects, the sets of biomarkers are processed using an algorithm. The algorithm may be a trained algorithm. The trained algorithms may use the sets of biomarkers as an input and generate an output regarding the presence or absence of a cancer. The output may be specific to a type of cancer or subtype of cancer. For example, the output may indicate the presence of a castrate-resistant prostate cancer.

The trained algorithm may be trained on multiple samples. For example, the trained algorithm may be trained using at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 300, 400, 500, 600,700, 800, 900, 1000, 1100, 1200, 1300, 1400, 1500, 1600, 1700, 1800, 1900, 2000, 3000, 4000, 5000, 6000, 7000, 8000, 9000, 10000, or more independent training samples. The trained algorithm may be trained using no more 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 300, 400, 500, 600,700, 800, 900, 1000, 1100, 1200, 1300, 1400, 1500, 1600, 1700, 1800, 1900, 2000, 3000, 4000, 5000, 6000, 7000, 8000, 9000, 10000, or less, independent training samples. The training samples may be associated with a presence or an absence of said cancer. The training samples may be associated with a relapse of cancer. The training samples may be associated with cancer that is resistant to a particular drug or treatment. An individual training sample may be positive for a particular cancer. An individual training sample may be negative for a particular cancer. By using training samples, the trained algorithm may be able to detect a cancer, determine a probability of recurrence or relapse of a cancer, or determine if a cancer comprises a set of biomarkers may be resistant to a treatment. The training sample may be associated with additional clinical health data of a subject. For example, additional clinical health data may comprise the gender, weight, height, or levels of metabolites or antibodies in a subjects. Additional clinical health data may comprise indication of other diseases, disorders, or diseases conditions.

The trained algorithms may be trained using multiple sets of training samples. The sets may comprise training samples as described elsewhere herein. For example, the training may be performed using a first set of independent training samples associated with a presence of said cancer and a second set of independent training samples associated with an absence of said cancer. Similarly, a first set may be associated with relapse and a second sample may be associated with the absence of relapse.

The trained algorithm may also process additional clinical health data of the subject. For example, additional clinical health data may comprise the gender, weight, height, or levels of metabolites or antibodies in a subjects. Additional clinical health data may comprise indication of other diseases, disorders, or diseases conditions that the subject may suffer from. By using the additional clinical health data, in conjunction with the biomarkers, the trained algorithm may output a presence or absences of cancer, probability of relapse, or resistance to drug treatment, that may be different from the output of an algorithm that does not process additional clinical health.

The trained algorithm may be an unsupervised machine learning algorithm. For example, the unsupervised machine learning algorithm may utilize cluster analysis to identify attributes of interest. The trained algorithm may be a supervised machine learning algorithm. For example, the algorithm may be inputted with training data such to generate an expected or desired output. The supervised learning algorithm may comprise a deep learning algorithm, a support vector machine (SVM), a neural network, or a Random Forest. Via the machine learning algorithm, the trained algorithm may be able to identify relationships of biomarkers to a particular cancer prognosis or diagnosis. Without the trained algorithm, it may otherwise be difficult to identify relationships of the biomarkers to accurately identify the presence of a cancer or other parameters associated with the cancer.

In various aspects, the systems and methods may comprise a accuracy, sensitivity, or specificity of detection of the cancer or a parameter of the cancer. For example, the methods or systems may comprise detecting the presence or the absence of cancer (or the presence of a parameter of the cancer, such as recurrence, relapse, or drug resistance) in the subject at an accuracy of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a sensitivity of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a specificity of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a positive predictive value of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a negative predictive value of at least about 60%, at least about 70%, at least about 75%, at least about 80%, at least about 85%, at least about 90%, at least about 95%, at least about 98%, or at least about 99%.

### Computer Systems

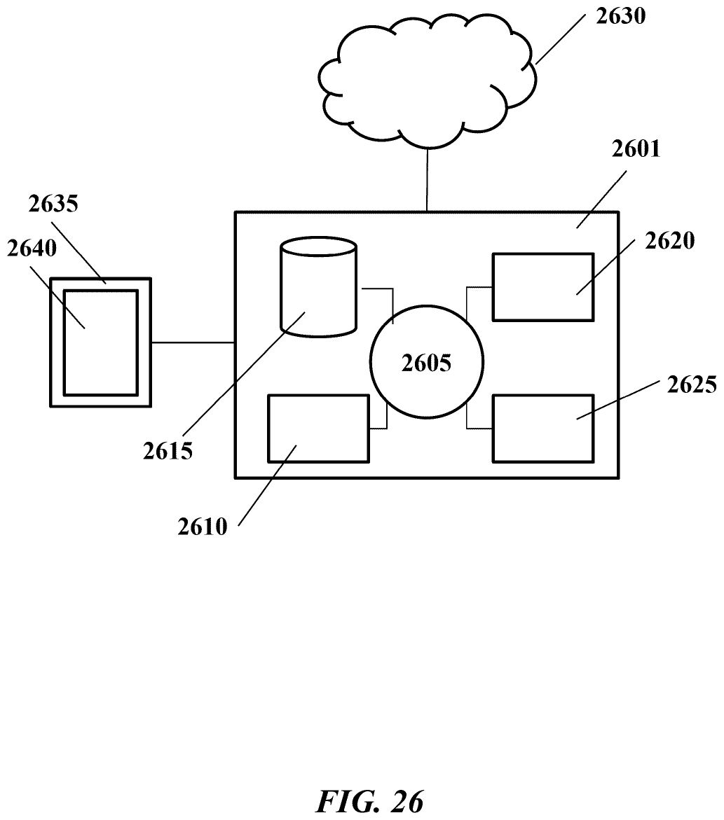

The present disclosure provides computer systems that are programmed to implement methods of the disclosure. FIG. 26 shows a computer system 2601 that is programmed or otherwise configured to perform analysis or steps of the methods, for example determine a likelihood of the presence of a cancer based on a set of biomarkers of an individual or run an algorithm. The computer system 2601 can regulate various aspects of methods and systems of the present disclosure, such as, for example, perform an algorithm, input training data, analyze sets of biomarker, or output a result for the user as to the presence or absence of cancer. The computer system 2601 can be an electronic device of a user or a computer system that is remotely located with respect to the electronic device. The electronic device can be a mobile electronic device.

The computer system 2601 includes a central processing unit (CPU, also “processor” and “computer processor” herein) 2605, which can be a single core or multi core processor, or a plurality of processors for parallel processing. The computer system 2601 also includes memory or memory location 2610 (e.g., random-access memory, read-only memory, flash memory), electronic storage unit 2615 (e.g., hard disk), communication interface 2620 (e.g., network adapter) for communicating with one or more other systems, and peripheral devices 2625, such as cache, other memory, data storage and/or electronic display adapters. The memory 2610, storage unit 2615, interface 2620 and peripheral devices 2625 are in communication with the CPU 2605 through a communication bus (solid lines), such as a motherboard. The storage unit 2615 can be a data storage unit (or data repository) for storing data. The computer system 2601 can be operatively coupled to a computer network (“network”) 2630 with the aid of the communication interface 2620. The network 2630 can be the Internet, an internet and/or extranet, or an intranet and/or extranet that is in communication with the Internet. The network 2630 in some cases is a telecommunication and/or data network. The network 2630 can include one or more computer servers, which can enable distributed computing, such as cloud computing. The network 2630, in some cases with the aid of the computer system 2601, can implement a peer-to-peer network, which may enable devices coupled to the computer system 2601 to behave as a client or a server.

The CPU 2605 can execute a sequence of machine-readable instructions, which can be embodied in a program or software. The instructions may be stored in a memory location, such as the memory 2610. The instructions can be directed to the CPU 2605, which can subsequently program or otherwise configure the CPU 2605 to implement methods of the present disclosure. Examples of operations performed by the CPU 2605 can include fetch, decode, execute, and writeback.

The CPU 2605 can be part of a circuit, such as an integrated circuit. One or more other components of the system 2601 can be included in the circuit. In some cases, the circuit is an application specific integrated circuit (ASIC).

The storage unit 2615 can store files, such as drivers, libraries and saved programs. The storage unit 2615 can store user data, e.g., user preferences and user programs. The computer system 2601 in some cases can include one or more additional data storage units that are external to the computer system 2601, such as located on a remote server that is in communication with the computer system 2601 through an intranet or the Internet.

The computer system 2601 can communicate with one or more remote computer systems through the network 2630. For instance, the computer system 2601 can communicate with a remote computer system of a user (e.g., a medical professional or patient). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2601 via the network 2630.

Methods as described herein can be implemented by way of machine (e.g., computer processor) executable code stored on an electronic storage location of the computer system 2601, such as, for example, on the memory 2610 or electronic storage unit 2615. The machine executable or machine readable code can be provided in the form of software. During use, the code can be executed by the processor 2605. In some cases, the code can be retrieved from the storage unit 2615 and stored on the memory 2610 for ready access by the processor 2605. In some situations, the electronic storage unit 2615 can be precluded, and machine-executable instructions are stored on memory 2610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 2601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2601 can include or be in communication with an electronic display 2635 that comprises a user interface (UI) 2640 for providing, for example, an input of biomarkers or sequencing data, or an visual output relating to a detection, diagnosis, or prognosis. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2605. The algorithm can, for example, determine a presence or absence of a cancer or cancer parameter based on a set of input sequencing data from a sample derived from a subject.

### EXAMPLES

### Example 1: Analysis of Cell Free DNA and Germline DNA for Detection of Cancer

Using methods and systems of the present disclosure, circulating tumor DNA-based alterations were detected in subjects with metastatic hormone-sensitive and castrate-resistant prostate cancer. These results are described by, for example, Kohli et al., “Clinical and genomic insights into circulating tumor DNA-based alterations across the spectrum of metastatic hormone-sensitive and castrate-resistant prostate cancer,” EBioMedicine 54 (2020), doi.org/10.1016/j.ebiom.2020.102728, which is incorporated by reference herein in its entirety.

Samples from multiple subjects with metastatic prostate cancer, including those with metastatic castrate resistant prostate cancer, were obtained. 4 separate groups of subject were analyzed. The first group, “Untreated metastatic hormone sensitive-prostate cancer (mHSPC),” included mHSPC patients whose first sample collection was performed before androgen deprivation treatment (ADT) initiation. Several, but not all, patients in this group had a second serial blood sample collection after 3 months of ADT; these serially collected patients were labeled the “3-month mHSPC” subgroup. The second group, “mHSPC on ADT,” included patients who were enrolled at the time of the first sample collection while undergoing continuous ADT. These patients had a biochemical response confirmed by stable to lower serum prostate-specific antigen (PSA) levels compared to pre-ADT initiation levels. The third independent group, “Biochemical progressive metastatic castrate resistant prostate cancer (mCRPC),” included patients with biochemical progression on ADT (defined as serially rising PSA levels above a previous PSA nadir) and castrate testosterone levels at the time of first blood sample collection and before a secondary hormonal maneuver or any additional new drug was administered for progression. No evidence of radiographic progression was observed in these patients. The fourth group, “Clinical mCRPC,” consisted of mCRPC patients with both biochemical failure (rising serial PSA levels above a previous nadir) and appearance of new radiographic metastases, with the sample collection performed before any new drug addition for progressive disease.

Ten milliliters of plasma separator tubes using K2-ethylenediamine tetraacetic acid (EDTA) and 3.2% buffered sodium citrate anticoagulants were used for specimen collections. Collected blood was initially centrifuged at 3000 rpm for 10 minutes at 4° C. The supernatant was then removed, and a second centrifugation was performed at 3000 rpm for 10 minutes. The platelet-poor plasma was fractioned into multiple aliquots for storage at −80° C., with no aliquots undergoing freeze-thaw cycles prior to cfDNA isolation. Circulating cfDNA was extracted using a QIAamp Circulating Nucleic Acid Kit from EDTA and citrate anticoagulant plasma. Germline DNA (gDNA) was extracted from matched peripheral blood mononuclear cells collected at the same time as plasma. The extracted cfDNA and gDNA (˜5-˜30 ng of cfDNA and ˜40 ng of gDNA per unique patient sample) was end-repaired before dA-tailing process, and then ligated with Unique Molecular Identifier (UMI) adapters. The DNA was allowed to hybridize to a set of sequence specific biotin-labeled probes in order to enrich for specific DNA. Unbound fragments were washed and the remaining DNA fragments were amplified via PCR. The resulting DNA library was sequenced on a HiSeq XTen sequencer with paired-end 2×150 bp sequencing kits.

The sequencing data from the samples was analyzed by using cleaned paired FASTQ files with outputs and aligned to human reference genome build hg19 using Burrows-Wheeler Alignments. Additionally the data was analyzed by generating consensus binary alignment map (BAM) files derived by merging paired-end reads that originated from the same molecules (based on mapping location and unique molecular identifiers) as single strand fragments. Single-strand fragments from the same double-strand DNA molecules were merged to be double stranded for suppressing sequencing and PCR errors. NGS quality-checking was performed by examining the percentage of targeted regions with >1500× unique consensus coverage. Samples with <80% regions having >1500× unique coverage were deemed to be QC failed and excluded. Candidate variants, consisting of point mutations, small insertions and deletions, were identified using the in-house developed pipeline across the targeted regions and comparing with local variant background. Variants were further filtered by log-odds (LOD) thresholds, base quality and mapping quality thresholds, repeat regions and other quality metrics.

The on-target unique fragment coverage was calculated on the basis of consensus sequence from BAM files; the fragment was also corrected for GC bias. The GC-adjusted unique fragment was then compared against corresponding coverage from a group of normal reference samples to estimate the significance of the copy number variant. Amplification or deletion of a gene copy number with an absolute z-score >2·58 (the inverse of the cumulative distribution function of a Gaussian distribution at 99.5% confidence level) were deemed as true events. ctDNA fraction was estimated on the basis of the allele fractions of autosomal somatic mutations. Mutant allele fraction (MAF) and the ctDNA fraction were related based on the formula ctDNA=2/([1/MAF]+1). Somatic mutations in genes with a detectable copy number amplification were omitted from ctDNA fraction estimates. ctDNA fractions were then estimated as the product of ctDNA fraction and total cfDNA yield in the corresponding sample following a normalization by plasma volume. Plasma tumor mutational burden (pTMB) was calculated as the number of somatic coding SNVs, including synonymous and nonsynonymous variants detected in the plasma samples after removing germline single-nucleotide polymorphisms.

DNA yield and ctDNA fraction and the number of variants in the coding regions of the genes covered by the panel (denoted as pTMB) was calculated for all subjects in the 4 groups relating to the and compared the overall group and intergroup-wise distributions for differences as shown in Table 3.

TABLE 3

| | | Serial 3-month | mHSPC on | Biochemical | |
|---|---|---|---|---|---|
| | Untreated mHSPC | mHSPC group | ADT group | Progressive CRPC | Clinical mCRPC |
| | group (n = 73) | (n = 29/73) | (n = 33) | group (n = 75) | group (n = 69) |
| Median cfDNA, | 4.32 (1.58-212.88) | 4.2 (1.78-10.67) | 3.7 (1.6-103.56) | 12.26 (3.87-84.71) | 17.97 (5.54-678.86) |
| ng/ml (range) | | | | | |
| Group | Untreated vs | 3-month post ADT | Untreated | Untreated mHSPC | Group comparison |
| comparison of | 3-month ADT | vs continuous ADT | mHSPC vs | vs clinical CRPC | of cfDNA yields, |
| cfDNA yields, | treated (P = .22) | (P = .83) | biochemical | (P < .001) | type (P value) |
| type (P value) | (N = 29) | | CRPC relapse | | |
| | | | (P < .001) | | |
| Median ctDNA | 0.01(0.006-0.63) | 0.009 (0.006-0.23) | 0.01(0.006-0.92) | 0.01(0.006-0.76) | 0.04(0.006-0.75) |
| fraction (range) | | | | | |
| Group | Untreated vs | 3-month post ADT | Untreated | Untreated mHSPC | Group comparison |
| comparison of | 3-month ADT | vs continuous ADT | mHSPC vs | vs clinical CRPC | of ctDNA fraction, |
| ctDNA fraction, | treated (P = .02) | (P = .79) | biochemical | (P < .001) | type (P value) |
| type (P value) | (N = 29) | | CRPC relapse | | |
| | | | (P < .001) | | |
| pTMB | 3 (1-28) | 4 (1-12) | 3 (1-13) | 5 (1-41) | 5 (1-28) |
| variants ^{a}, no. | | | | | |
| pTMB-based | Untreated vs | 3-month post ADT | Untreated | Untreated mHSPC | pTMB-based group |
| group | 3-month ADT | vs continuous ADT | mHSPC vs | vs clinical CRPC | comparisons, |
| comparisons, | treated (P = .18) | (P = .47) | biochemical | (P < .001) | type (P value) |
| type (P value) | (N = 29) | | CRPC relapse | | |
| | | | (P < .001) | | |
| PTMB-based group | NA | NA | NA | P < .001 | pTMB-based group |
| comparisons for | | | | | comparisons for |
| mHSPC state vs. | | | | | mHSPC state vs. |
| mCRPC, P value | | | | | mCRPC, P value |

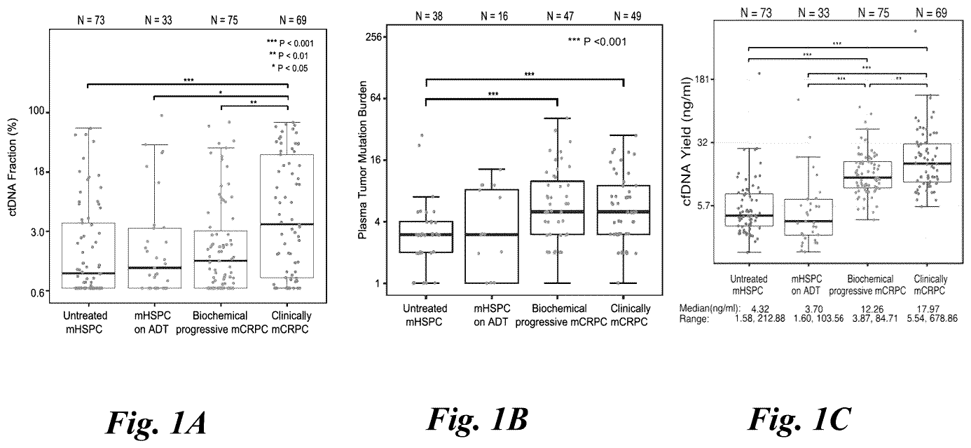

The distributions for and comparisons between them are also shown in FIG. 1A (ctDNA), and FIG. 1B (pTMB), and FIG. 1C(cfDNA). The cfDNA yield/ctDNA fraction and pTMB levels were significantly greater in the mCRPC groups than in the mHSPC groups (P<·001, Kruskal-Wallis test). There were no noticeable differences in cfDNA yield/ctDNA fraction or in the pTMB levels between untreated mHSPC and mHSPC on ADT groups.

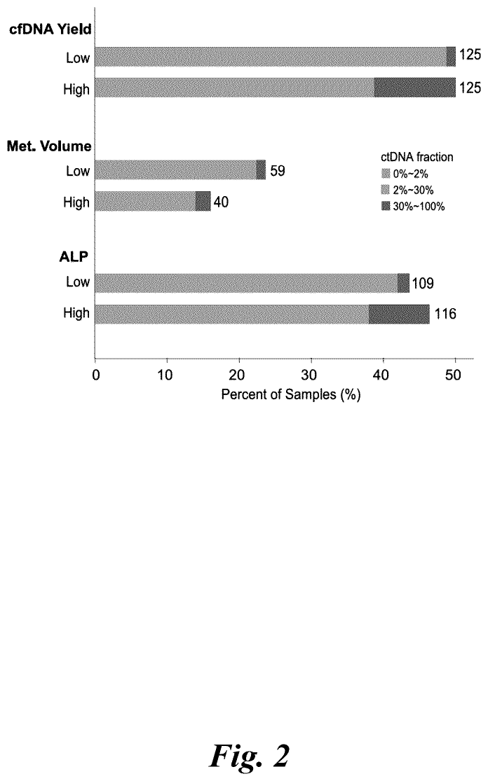

A median cfDNA yield cutoff value of 9.6 ng/mL was used for all study samples based on which the ctDNA fraction distribution was determined (top panel of FIG. 2). A definition of high- and low-volume metastatic disease was used to stratify high vs low metastatic volume in the untreated metastatic hormone-sensitive group. FIG. 2 (middle panel) shows the distribution of ctDNA fractions in high- and low-volume metastatic disease. The lower panel in FIG. 2 shows ctDNA fraction distributions above and below the median serum alkaline phosphatase (ALP) levels (median, 83 IU/L), a known prognostic factor for survival in castration-resistant state.

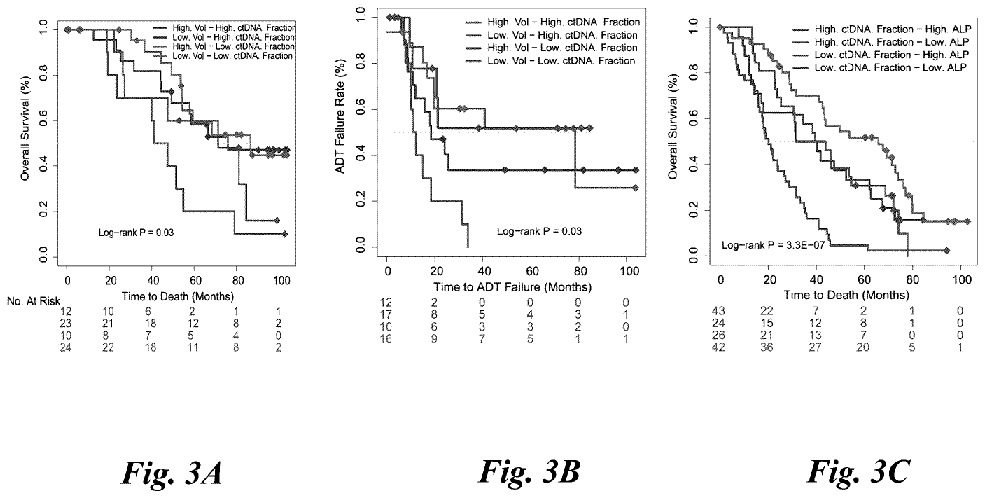

The cfDNA yield/ctDNA fraction and pTMB levels were calculated to be used as a predictive value of these variables for ADT efficacy in patients in the untreated mHSPC group using ADT failure time and assessed their prognostic value for overall survival (OS) in patients in mHSPC and mCRPC states. Subjects with high-volume metastatic disease in the untreated mHSPC group had the shortest OS, however, metastatic volume was not predictive of the duration of ADT failure. The combined effect of cfDNA yield/ctDNA fraction and metastatic disease volume on survival was analyzed. Untreated mHSPC patients with high-volume metastatic disease and high-yield cfDNA/ctDNA had the shortest OS, and patients with low-volume metastatic disease and low cfDNA yield/ctDNA fraction had the longest OS. Interestingly, not all patients with high-volume metastatic disease had poor OS, as a group of mHSPC patients with high-volume metastatic disease and low nucleic acid yields had intermediate OS similar to those of patients with low-volume metastatic disease and high cfDNA yield/ctDNA fraction. FIG. 3A shows the Kaplan-Meier plots for OS based on volume status and ctDNA fraction in the untreated mHSPC group. FIG. 3B shows the combined effect of ctDNA fraction and metastatic disease volume on ADT failure rates; patients with high-volume metastases and high ctDNA fraction exhibited the shortest time to ADT failure. The prognostic value of ALP levels on OS, a known clinical prognostic factor in mCRPC, was determined and nucleic acid yield/fraction-based prognosis evaluated. The combined effect of ALP and nucleic acid yield/fraction on OS for all mCRPC patients is shown in FIG. 3C for ctDNA

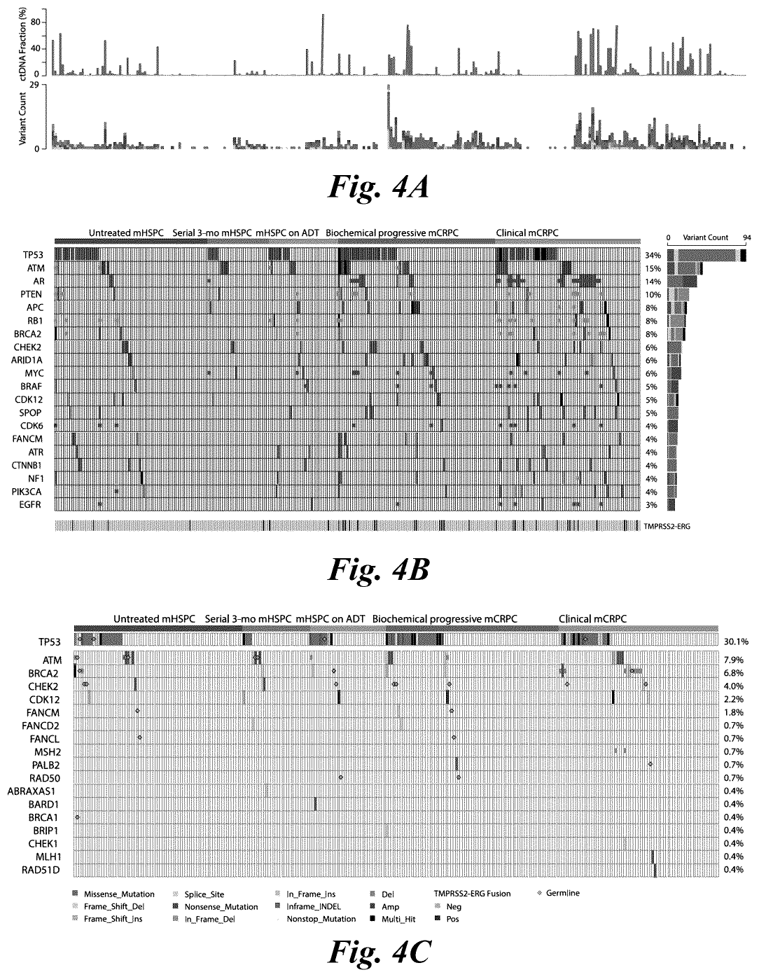

A spectrum of alterations was characterized in each of the 4 independent groups of mHSPC and mCRPC patients. A 120 gene panel was used to identify ctDNA-based CNAs, SNVs, and the frequency of TMPRSS2-ERG fusion. FIG. 4 shows the individual patient ctDNA fractions and variant counts (FIG. 4A) of all patients. FIG. 4B shows the overall distribution of plasma ctDNA somatic alterations in the top 26 genes across the spectrum of all 4 groups (n=250) that were altered in ≥3% of the study patients using the targeted 120 gene panel. FIG. 4C represents deleterious/likely deleterious alterations detected in genes that were involved in DNA damage repair (DDR) pathways and includes copy number loss and germline pathogenic mutations. Copy number loss is shown as a blue box, and germline deleterious/likely deleterious mutations are marked with a green diamond shape.

Table 4 describes the number of patients in each metastatic group who had a genomic alteration of any kind and shows the intergroup comparisons that were performed. All 3 types of somatic alterations (SNVs, CNAs, and TMPRSS2-ERG fusions) were detected more frequently in mCRPC patients than in mHSPC patients. Within the mCRPC groups, a significantly higher proportion of clinical mCRPC group patients had somatic events compared to all other groups

TABLE 4
Patients with NGS-analyzable data with plasma ctDNA based detectable
alterations in different metastatic prostate cancer groups

| | | SNVs | | | | CNAs | | | | Fusions | | | |
|---|---|---|---|---|---|---|---|---|---|---|---|---|---|
| | Total | Patients | | | Adjusted | Patients | | | | Patients | | | |
| Metastatic | Patients, | detected, | | P | P | detected, | | P | Adjusted | detected, | | P | Adjusted |
| Groups | no. | no (%) | OR | value¹ | value | no (%) | OR | value¹ | P value | no (%) | OR | value | P value |
| Untreated | 73 | 50 (68) | — | — | — | 6 (8) | — | — | — | 4 (6) | — | — | — |
| mHSPC | | | | | | | | | | | | | |
| state | | | | | | | | | | | | | |
| Serial 3- | 29 | 22 (76) | 1.44 | .63 | .63 | 1 (3) | 0.40 | .67 | .89 | 1 (3) | 0.62 | 1.00 | 1.0 |
| month | | | | | | | | | | | | | |
| mHSPC | | | | | | | | | | | | | |
| subgroup | | | | | | | | | | | | | |
| mHSPC | 33 | 25 (76) | 1.43 | .50 | .63 | 3 (9) | 1.12 | 1.00 | .00 | 2 (6) | 1.11 | 1.00 | 1.0 |
| on ADT | | | | | | | | | | | | | |
| Biochemical | 75 | 59 (79) | 1.69 | .19 | .39 | 9 (12) | 1.52 | .59 | .89 | 13 (17) | 3.59 | .04 | .07 |
| progressive | | | | | | | | | | | | | |
| mCRPC | | | | | | | | | | | | | |
| Clinical | 69 | 62 (90) | 4.04 | .002 | .008 | 25 | 6.26 | 7.4^{x-05} | .0004 | 14 (20) | 4.35 | .01 | .04 |
| mCPRC | | | | | | (36) | | | | | | | |

| | | | |
|---|---|---|---|
| mHSPC¹ (n = 106) | P = .01 | P = .002 | P = .002 |
| vs. mCRPC² | | | |
| (n = 144) | | | |

FIGS. 4B and 4C also shows TP53, AR, DDR pathway genes, cell cycle control and differentiation pathway genes, and well-known tumor suppressor genes to be among the most frequent genes within the top 20 genes with detectable somatic alterations. An increased number of genes in mCRPC patients (n=33 [23%]) had detectable CNAs (amplifications/deletions) compared to mHSPC patients (n=8 [8%]). AR gene amplification was the most common CNA and was largely detected in the mCRPC group patients. EGFR, MYC, BRAF, and CDK6 gene amplifications were detected in both mHSPC and mCRPC patients. RB1 deletion/loss was the most frequently detected deletion/loss, and 41/250 patients also had copy number loss of DNA repair pathway genes (FIG. 2C) in both the mHSPC (n=14/106) and mCRPC groups (n=27/144). An increased number of mCRPC patients (n=33 [23%]) had detectable CNAs (amplifications/deletions) compared to mHSPC patients (n=8 [8%]).

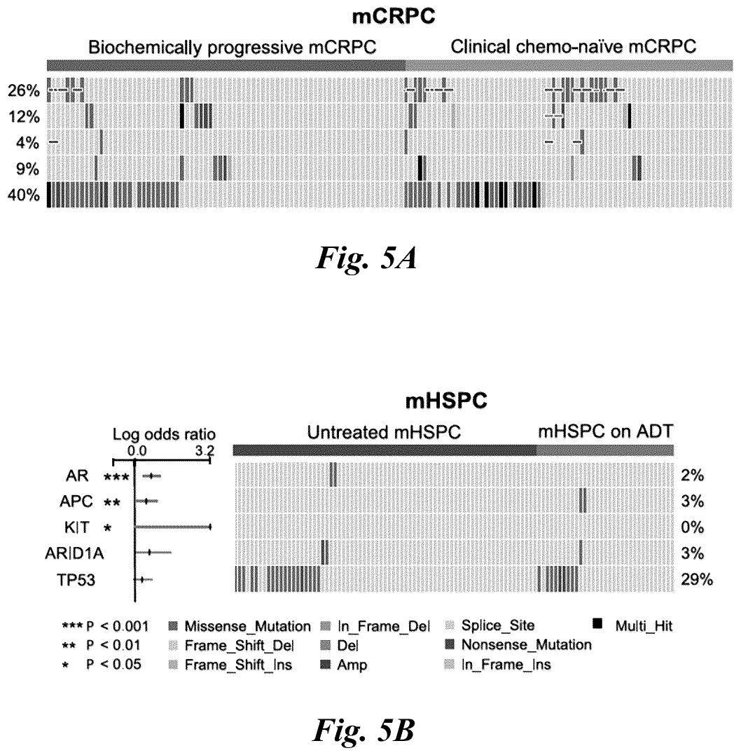

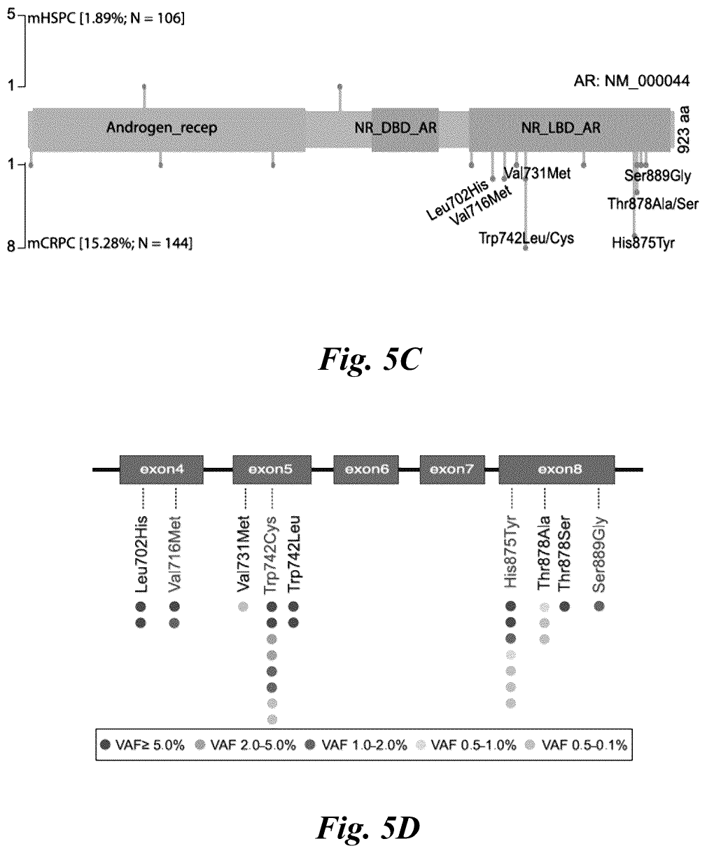

The overall frequency of ctDNA mutations, which were significantly increased in patients in the mCRPC groups compared to patients in the mHSPC groups (FIGS. 5A and 5B), were observed in AR, APC, and KIT genes (P<·05). AR hotspot mutations were detectable in patients in the mCRPC groups. FIG. 5C shows that these mutations were in the ligand-binding domain of the receptor and indicates the number of patients with each mutation. Mutations T742L, T742C, V716M, T878A, L702H, H875Y and other novel hotspot AR mutations were among those detected in patients in the mCRPC groups. FIG. 5D further shows the distribution of AR hotspot mutations across exon regions in mCRPC patients at the different levels of variant allelic frequency of detection. Each dot represents a patient, and the distinct colors indicate different levels of variant allelic frequency (VAF). FIG. 5E shows the per-patient occurrence of detectable AR mutations, AR copy number gain, and individual patient-level ctDNA fractions in both mCRPC groups. Each colored bar represents an individual patient.

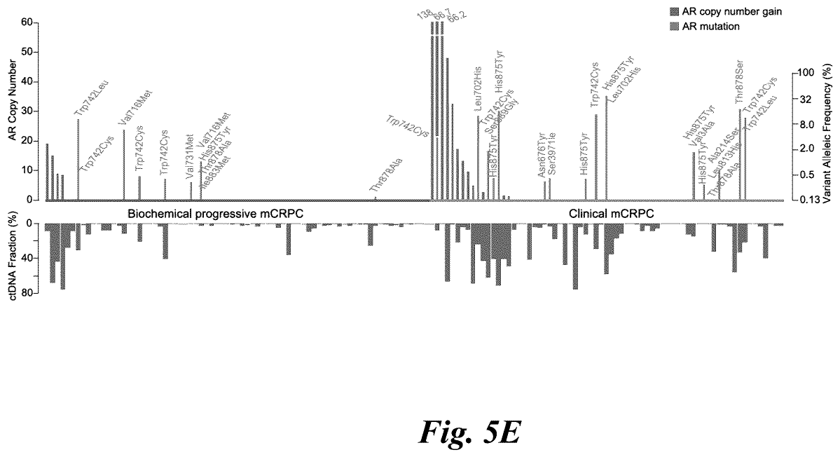

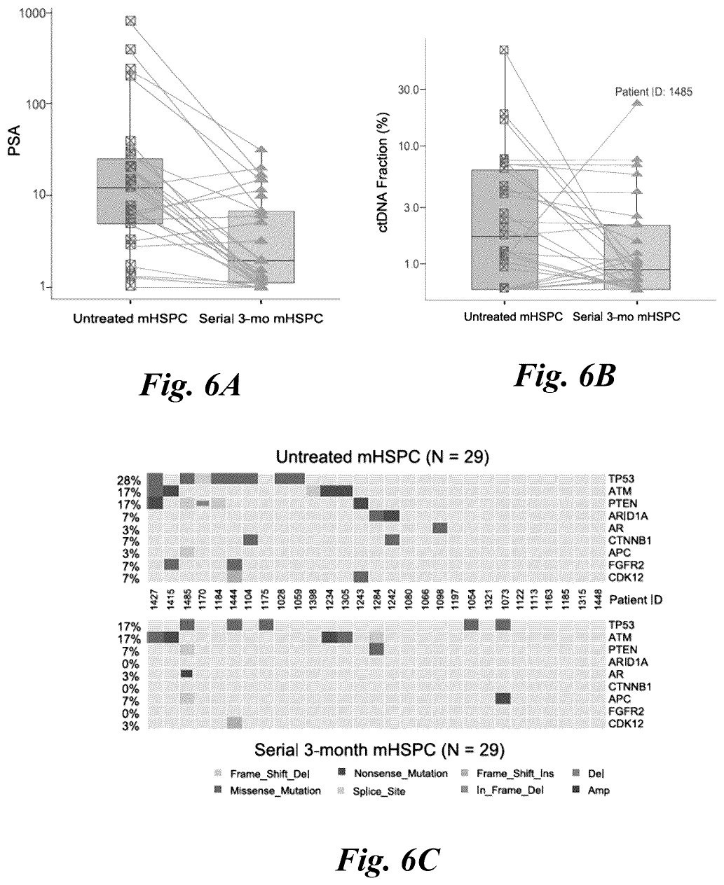

Pharmacodynamic changes in ctDNA-based alterations were interrogated before and after 3 months of ADT in the 29 paired plasma collections of the untreated mHSPC group patients (n=29; [40%]). Changes in PSA (FIG. 4A), ctDNA fractions (FIG. 6B) and specific clonal changes of the most frequently mutated genes before and after 3 months of ADT treatment in the untreated mHSPC group are shown in FIG. 4C. After 3 months of ADT, several patients demonstrated a PSA response but not a complete disappearance of plasma ctDNA fraction and/or detectable mutations (FIG. 6B). Appearance of new mutations after 3 months of ADT with PSA response was also observed (FIG. 6C: patients 1175, 1284, 1054, and 1073).

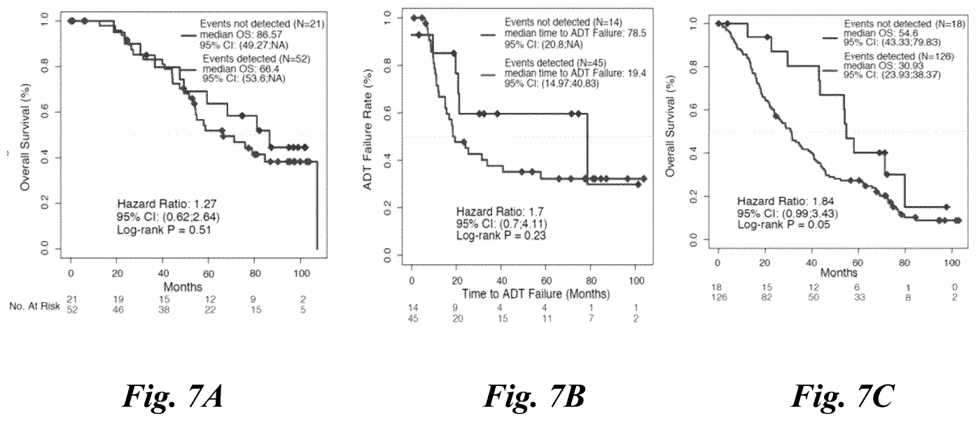

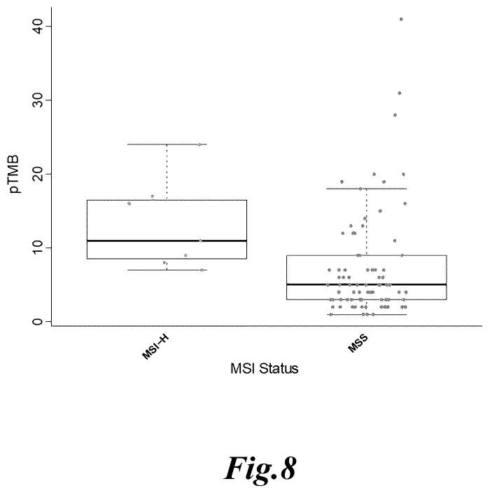

Presence of detectable somatic events did not affect ADT failure rates (FIG. 7B). In comparison, in the mCRPC groups, patients not detected to have somatic events had a significantly longer OS than mCRPC patients with detected events (54.6 vs 30.93 months; HR, 1.84 [95% CI, 0.99-3.43]; Log-Rank P=. 05) (FIG. 7C). MSI status was also observed and correlation with plasma TMB in mHSPC/mCRPC patients detected with MMR-deficiency mutations (and/or MSI-high status, hypermutation) were analyzed (FIG. 8).

OS outcomes were also determined for the mHSPC and mCRPC groups on the basis of individual-gene and multiple-gene alterations after adjusting for known prognostic variables in both groups. In untreated mHSPC patients at the individual-gene level, alterations in TP53 and ATM were significantly associated with shorter OS. These alterations were not significant after adjusting for metastatic volume and Gleason Score. Collectively, untreated mHSPC patients with somatic alterations detected in multiple DNA repair genes (ATM, BRCA1, BRCA2, and CHEK2) had significantly short OS (HR, 4·0 [95% CI, 1·4-11·8]; P=4·75×10-05) (Table 5 and a shorter time to failure with ADT even after adjusting for clinical prognostic factors.

TABLE 5
Overall survival and time to ADT failure based on ctDNA alteration
events in the untreated mHSPC group at the univariate level after adjusting for ctDNA
fraction, metastatic volume and Gleason score.

| | Patients | Overall Survival | | | | | | |
|---|---|---|---|---|---|---|---|---|
| | with | | Univariate | | | Multivariate | | |
| | events, | Median, | HR (95% | P | Adj. P | HR (95% | P | Adj. P |
| Gene | no. | mo | CI) | value | value | CI) | value | value |
| TP53 | 19 | 54.4 | 2.0 (1.0- | .04 | .10 | 1.8 (0.8- | .13 | .26 |
| | | | 3.9) | | | 4.0) | | |
| ATM | 6 | 35.4 | 4.3 (1.6- | .002 | .006 | 2.8 (0.7- | .13 | .26 |
| | | | 11.5) | | | 11.0) | | |
| PTEN | 6 | 48.4 | 1.8 (0.6- | .33 | .47 | 0.8 (0.2- | .73 | .73 |
| | | | 5.0) | | | 3.5) | | |
| RB1 | 5 | 26.8 | 3.1 (0.9- | .05 | .10 | 0.5 (0.1- | .51 | .73 |
| | | | 10.2) | | | 4.0) | | |
| CTNNB1 | 4 | 79.0 | 0.9 (0.2- | .91 | .96 | 0.3 (0.1- | .11 | .26 |
| | | | 3.8) | | | 1.4) | | |
| SMO | 4 | 27.2 | 1.0 (0.2- | .96 | .96 | 0.4 (0.0- | .33 | .55 |
| | | | 4.3) | | | 2.8) | | |
| TMPRSS2-ERG | 4 | 68.0 | 1.4 (0.4- | .63 | .79 | 1.3 (0.4- | .68 | .73 |
| | | | 4.4) | | | 4.8) | | |
| TP53 + PTEN + | 23 | 54.4 | 1.7(0.9- | 0.10 | 0.17 | 1.2 (0.5- | .72 | .73 |
| RB1 | | | 3.4) | | | 2.5) | | |
| ATM + BRCA1/2 | 8 | 27.2 | 4.9 (2.0- | .0002 | .001 | 3.6 (1.1- | .04 | .20 |
| | | | 12.5) | | | 11.8) | | |
| ATM + BRCA1/2 + | 9 | 35.7 | 5.3 (2.2- | 4.75^{x-05} | .0005 | 4.3 (1.4- | .01 | .10 |
| CHEK2 | | | 12.8) | | | 13.5) | | |

Overall survival and time to ADT failure based on ctDNA alteration
events in the untreated mHSPC group at the univariate level after adjusting for ctDNA
fraction, metastatic volume and Gleason score.

| | Patients | Time to Treatment Failure | | | | | | |
|---|---|---|---|---|---|---|---|---|
| | with | | Univariate | | | Multivariate | | |
| | events, | Median, | HR (95% | P | Adj. P | HR (95% | P | Adj. |
| Gene | no. | mo | CI) | value | value | CI) | value | P value |
| TP53 | 19 | 15.0 | 2.4 (1.2- | .01 | .05 | 2.5 (1.1- | .02 | .20 |
| | | | 4.8) | | | 5.7) | | |
| ATM | 6 | 11.1 | 2.2 (0.8- | .12 | .21 | 1.2 (0.3- | .80 | .96 |
| | | | 6.5 | | | 4.6) | | |
| PTEN | 6 | 18.3 | 1.8 (0.6- | .25 | .36 | 1.0(0.3- | .96 | .96 |
| | | | 5.2) | | | 3.9) | | |
| RB1 | 5 | 11.1 | 1.4 (0.3- | .62 | .78 | 0.7 (0.1- | .72 | .96 |
| | | | 6.0) | | | 5.7) | | |
| CTNNB1 | 4 | 12.2 | 2.4 (0.7- | .13 | .22 | 1.1 (0.3- | .87 | .96 |
| | | | 8.1) | | | 4.5) | | |
| SMO | 4 | 11.1 | 0.8 (0.2- | .75 | .83 | 0.3 (0.0- | .26 | .78 |
| | | | 3.3) | | | 2.3) | | |
| TMPRSS2-ERG | 4 | 36.4 | 1.1 (0.3- | .84 | .84 | 0.8 (0.2- | .77 | .96 |
| | | | 3.7) | | | 3.6) | | |
| TP53 + PTEN + | 23 | 15.0 | 1.9 (1.0- | .06 | .15 | 1.4 (0.6- | .39 | .78 |
| RB1 | | | 3.7) | | | 3.1) | | |
| ATM + BRCA1/2 | 8 | 11.0 | 2.8 (1.1- | .03 | .10 | 1.7 (0.5- | .39 | .78 |
| | | | 7.5) | | | 5.9) | | |
| ATM + BRCA1/2 + | 9 | 11.0 | 3.4 (1.4- | .005 | .05 | 2.3 (0.7- | .16 | .78 |
| CHEK2 | | | 8.4) | | | 7.5) | | |

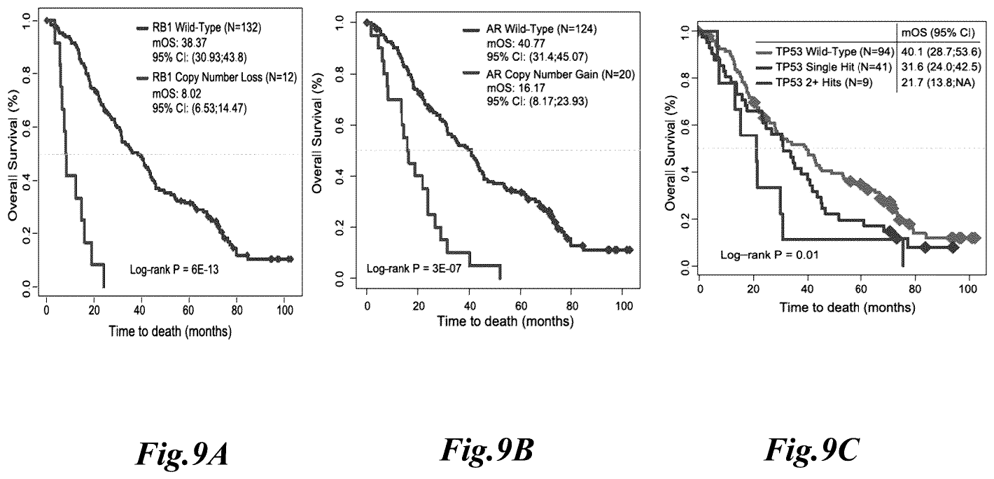

For the mCRPC groups, RB1 deletions had the most significant prognostic value for poor outcomes in multivariate analyses after adjusting for Gleason Score and ALP levels (HR, 4.2 [95% CI, 2.0-8.7]; P=1.5×−04). Somatic perturbations detected in other genes in multivariable analyses that had prognostic significance included TP53, CDH1, CDK6 and BRCA2 (Table 6). At the individual gene level, FIG. 9A shows RB1 copy number deletion to be associated with significantly worse OS in mCRPC patients. Similarly, FIG. 9B shows poor OS in mCRPC patients with AR copy number gain. mCRPC patients who harbored different TP53 mutations and an increasing number of mutations were also associated with poorer OS (FIG. 9C).

TABLE 6
Survival in mCRPC groups based on ctDNA SNVs, CNAs and fusion gene
events at the univariate and multivariate levels, adjusting for
ctDNA fraction, alkaline phosphatase levels and Gleason score

| | Patients | | Univariate | | |
|---|---|---|---|---|---|
| | with | Median | HR | P | Adjusted |
| Gene | events, no. | OS, mo | (95% CI) | value¹ | P value |
| TP53 | 50 | 30.8 | 1.5 (1.0-2.1) | .04 | .08 |
| AR | 37 | 20.1 | 2.3 (1.5-3.4) | 4.16×⁻⁰⁵ | 2.18×⁻⁰⁴ |
| TMPRSS2-ERG | 27 | 23.7 | 1.5 (1.0-2.4) | .05 | .10 |
| APC | 18 | 34.8 | 1.2 (0.7-2.0) | .51 | .67 |
| PTEN | 17 | 18.1 | 2.2 (1.3-3.7) | .003 | .01 |
| RB1 | 15 | 8.2 | 6.9 (3.8-12.6) | 2.18×⁻¹³ | 4.58×⁻¹² |
| BRCA2 | 13 | 13.7 | 1.5 (0.8-2.7) | .23 | .32 |
| ARID1A | 13 | 20.1 | 1.2 (0.6-2.2) | .63 | .73 |
| MYC | 11 | 15.9 | 1.5 (0.8-3.0) | .23 | .32 |
| ATM | 10 | 26.9 | 1.0 (0.5-2.1) | .90 | .90 |
| SPOP | 10 | 35.8 | 1.2 (0.6-2.4) | .61 | .73 |
| BRAF | 9 | 18.6 | 2.2 (1.1-4.3) | .02 | .05 |
| CDK6 | 9 | 10.2 | 2.9 (1.5-5.9) | .001 | .004 |
| NF1 | 8 | 38.1 | 0.8 (0.4-1.8) | .66 | .73 |
| PIK3CA | 8 | 16.1 | 2.3 (1.1-4.7) | .02 | .05 |
| ATR | 7 | 15.9 | 1.1 (0.5-2.5) | .80 | .84 |
| CDH1 | 7 | 9.5 | 7.3 (3.2-16.6) | 3.34×⁻⁰⁸ | 2.4×⁻⁰⁷ |
| EGFR | 7 | 26.6 | 2.3 (1.0-5.0) | .03 | .07 |
| TP53 + PTEN + RB1 | 63 | 24.0 | 2.0(1.4-2.9) | 1.50×⁻⁰⁸ | 1.58×⁻⁰⁷ |
| ATM + BRCA1/2 | 23 | 17.4 | 1.4 (0.8-2.3) | .22 | .32 |
| ATM + BRCA1/2 + CHEK2 | 24 | 18.9 | 1.4(0.9-2.3) | .17 | .30 |

| | |
|---|---|
| Patients | Multivariate |

| | with | Median | HR | P | Adjusted |
|---|---|---|---|---|---|
| Gene | events, no. | OS, mo | (95% CI) | value¹ | P value |
| TP53 | 50 | 30.8 | 1.6 (1.0-2.4) | .04 | .17 |
| AR | 37 | 20.1 | 1.3 (0.8-2.1) | .25 | .58 |
| TMPRSS2-ERG | 27 | 23.7 | 1.3 (0.8-2.1) | .35 | .61 |
| APC | 18 | 34.8 | 1.0 (0.6-1.8) | .95 | .98 |
| PTEN | 17 | 18.1 | 1.5 (0.8-2.8) | .19 | .50 |
| RB1 | 15 | 8.2 | 4.2 (2.0-8.7) | 1.5×⁻⁰⁴ | .003 |
| BRCA2 | 13 | 13.7 | 2.5 (1.3-5.1) | .01 | .07 |
| ARID1A | 13 | 20.1 | 0.9 (0.4-1.8) | .76 | .94 |
| MYC | 11 | 15.9 | 0.9 (0.4-2.2) | .83 | .96 |
| ATM | 10 | 26.9 | 0.9 (0.4-1.8) | .72 | .94 |
| SPOP | 10 | 35.8 | 1.2 (0.5-2.5) | .70 | .94 |
| BRAF | 9 | 18.6 | 1.1 (0.5-2.5) | .74 | .94 |
| CDK6 | 9 | 10.2 | 2.2 (1.0-4.6) | .05 | .18 |
| NF1 | 8 | 38.1 | 1.0 (0.5-2.2) | .98 | .98 |
| PIK3CA | 8 | 16.1 | 1.4 (0.6-3.0) | .44 | .71 |
| ATR | 7 | 15.9 | 1.8 (0.8-4.0) | .19 | .50 |
| CDH1 | 7 | 9.5 | 3.6 (1.2-10.8) | .02 | .11 |
| EGFR | 7 | 26.6 | 1.1 (0.5-2.6) | .87 | .96 |
| TP53 + PTEN + RB1 | 63 | 24.0 | 1.9 (1.3-2.9) | .002 | .02 |
| ATM + BRCA1/2 | 23 | 17.4 | 1.3 (0.8-2.3) | .32 | .61 |
| ATM + BRCA1/2 + CHEK2 | 24 | 18.9 | 1.3 (0.8-2.2) | .33 | .61 |

### Example 2: Analysis of Cell Free DNA and Germline DNA for Detection of Cancer

Using methods and systems of the present disclosure, combined cell-free DNA and RNA profiling was performed on the androgen receptor, which demonstrated clinical utility of a multianalyte liquid biopsy assay for metastatic prostate cancer. These results are described by, for example, Fettke et al., “Combined Cell-free DNA and RNA Profiling of the Androgen Receptor: Clinical Utility of a Novel Multianalyte Liquid Biopsy Assay for Metastatic Prostate Cancer,” European Association of Urology 78 (2020), pp. 173-180, which is incorporated by reference herein in its entirety.

Peripheral blood (10 ml) was collected in a single EDTA-containing or dedicated cfDNA-stabilizing tube (Streck, La Vista, Nebraska, USA) immediately prior to commencing systemic therapy (ARPIs or taxane chemotherapy). Two-step centrifugation was performed (1900 g for 10 min followed by 16 000 g for 10 min) to separate and clarify plasma and buffy coat (containing peripheral blood mononuclear cells [PBMCs]). Plasma and PBMCs were stored at 80 C until used for analysis. Briefly, PBMC-derived germline DNA (gDNA) and plasma cfDNA/cfRNA were extracted using a combination of kit and column-based methods. After quality assessment and quantification, 40 ng of fragmented gDNA, 5-30 ng of cfDNA, and up to 30 ng of fragmented cfRNA were used for library preparation, panel based hybridization (180-gene PredicinePLUS panel; Table 2), and enrichment prior to paired-end sequencing on the Illumina HiSeq XTen. GeneRADAR technology and DeepSea machine learning bioinformatics algorithm were then used to identify point mutations, insertions/deletions, splice site alterations, and copy number alterations. GeneRADAR incorporated ctDNA markers (CNAs, CNLs, SNVs, indels, rearrangements) and ctRNA markers(splicing, fusion, SNVs, indels). DeepSea machine learning platform processed sequence reads by a filtering of reads to remove low quality reads, performing error correction based on molecular barcode, performed consensus calling such to suppress sequencing/PCR errors, and integrated a knowledge database to generate high sensitivity and specificity and accurate variant calling.

Follow-up time was calculated from the date of sample acquisition to the date of last patient contact. AR aberrations were defined as AR copy number variation (ctDNA), AR somatic mutations (ctDNA), and AR-Vs (cfRNA), which were restricted to AR-V7 and AR-V9 due to their strong association with pathogenicity. Kaplan-Meier survival estimate (log-rank test) and multivariable Cox regression models (covariates: ctDNA fraction dichotomized into below or above 2%; prior taxane chemotherapy; prior ARPIs; performance status; presence of visceral metastases; and pain a enrollment) were then used to assess the association between ARaberrations and clinical outcomes, including (1) overall survival (OS; time from treatment commencement until death from any cause), (2)vprostate-specific antigen (PSA) response (PSA decline from baseline of 50%, confirmed 3 wk. later), (3) PSA progression-free survival (PSAPFS, as per Prostate Cancer Working Group 3 criteria and (4) clinical/radiographic progression-free survival (clin/rPFS). Evaluation of PSA response required 12 wk. of follow-up, and the 12-wk PSA response rate is reported. All three survival outcomes were censored at the date of last patient contact if the event had not occurred. Statistical significance was defined as p<0.05.

AR aberrations of any type were present in 36/67 (54%) patients at baseline; the distribution of AR aberrations is shown in FIG. 10. In FIG. 10, the asterisks denote ARPI therapy, whilst daggers denote taxane chemotherapy. Orange tiles represent presence of aberration; missing cfRNA data are denoted by grey tiles. AR copy number gain was found in 26/67 (39%) patients; of note, ctDNA fraction was not significantly higher in patients with AR copy number gain. AR somatic mutations were seen in 16/67 (24%) patients. Supplementary Table 7 of Fettke et al. summarizes the landscape of AR mutations, the most common of which was H875Y (n=10). The median allelic frequency of AR mutations was 2.1% (range 0.13-26%). Eleven (19%) and six (10%) patients harbored transcript expression of AR-V7 and AR-V9, respectively. As no patients exhibited all three types of AR aberrations, associations between cumulative AR aberrations and time-to-event outcomes were analyzed using a three-level model (zero/one/two or more aberrations).

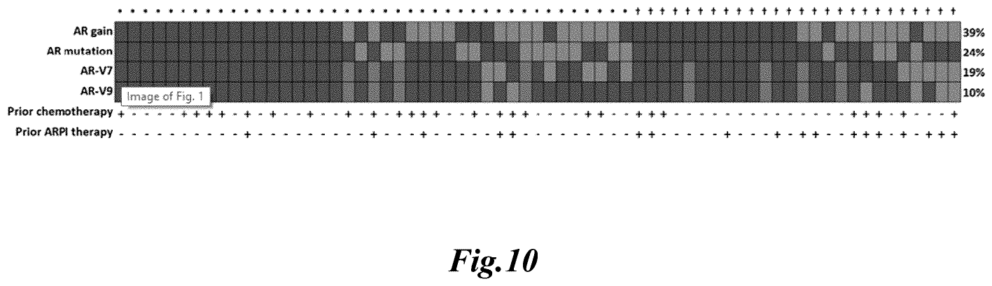

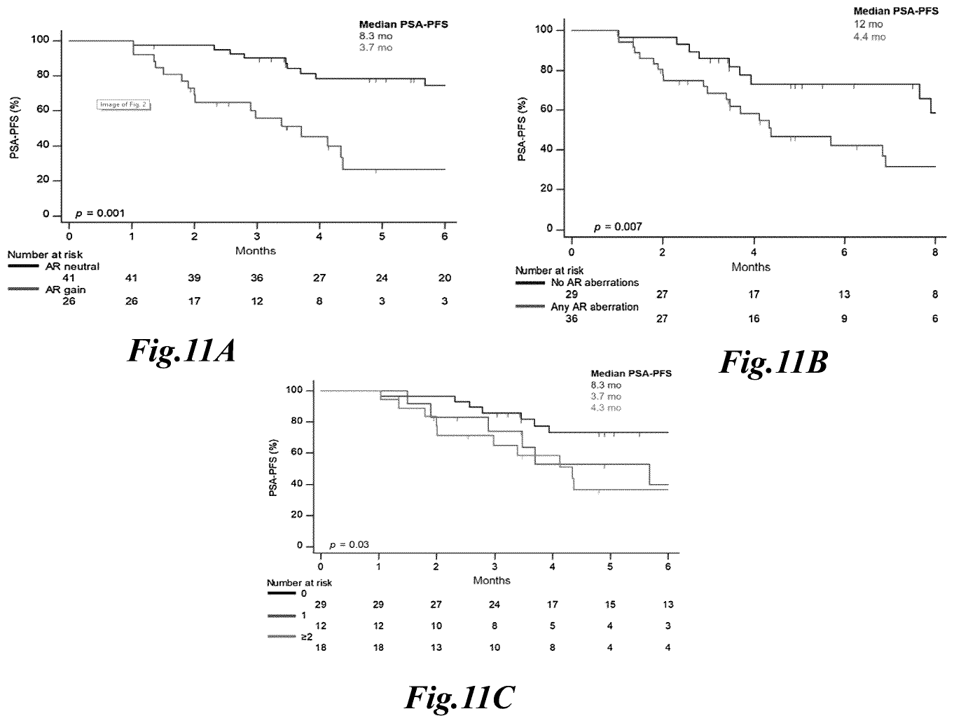

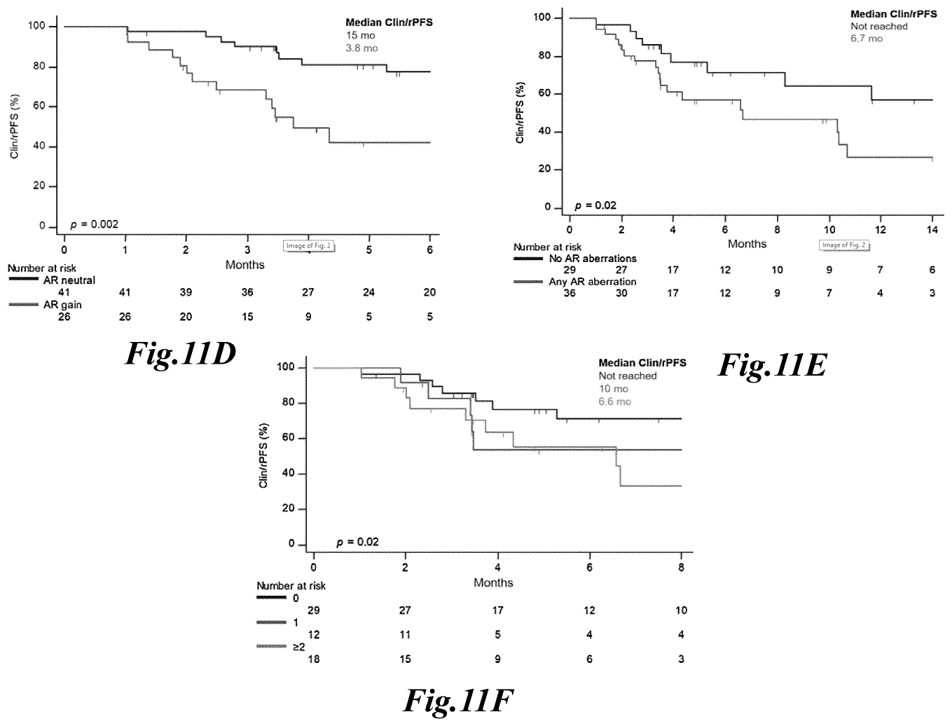

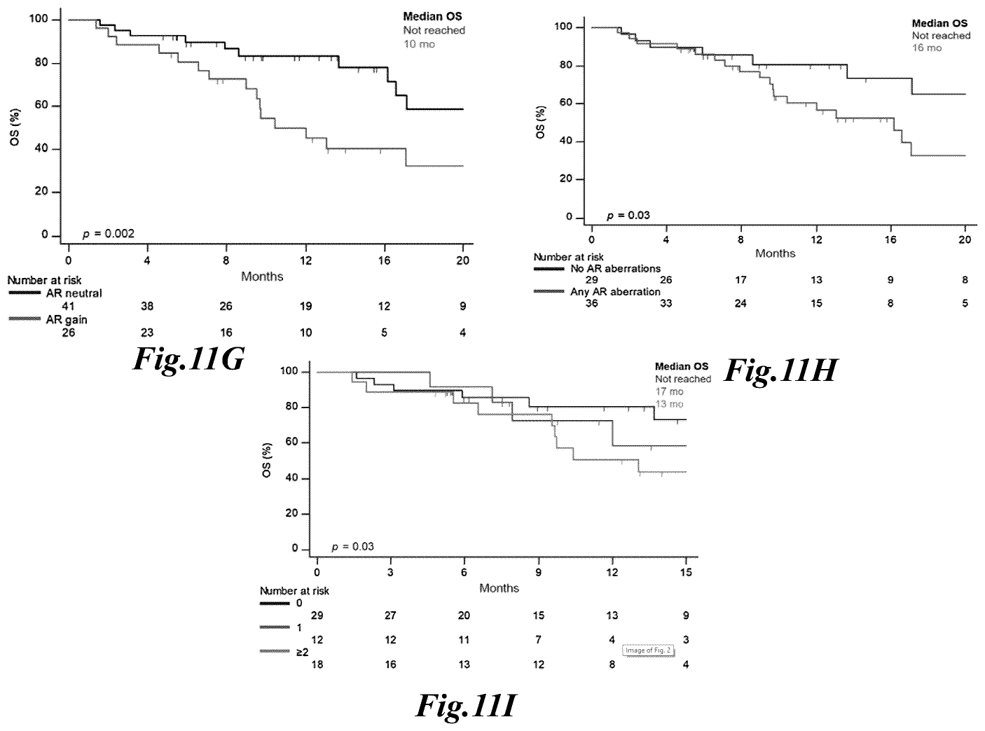

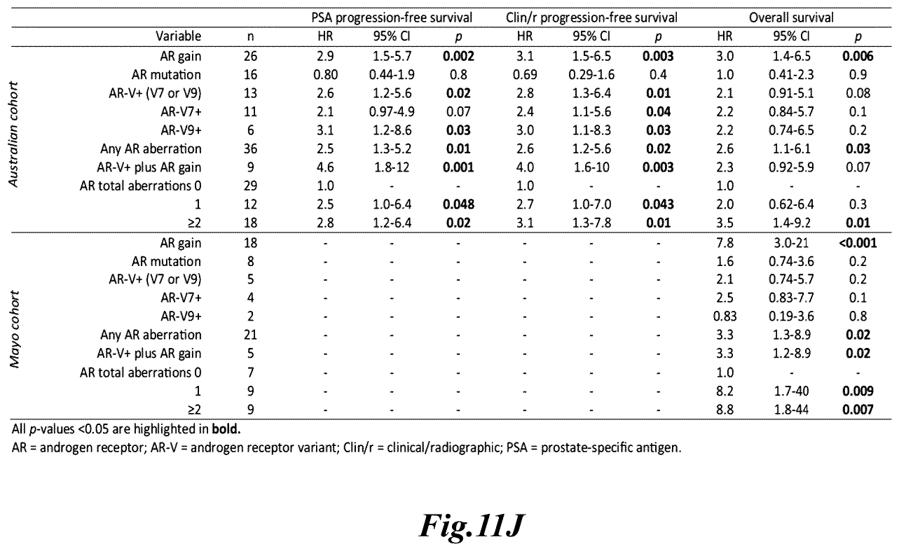

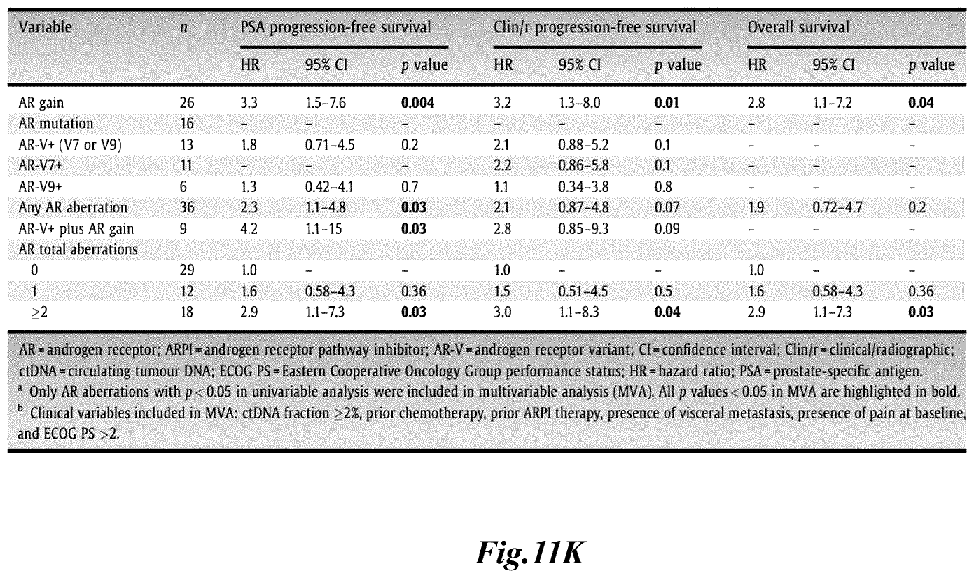

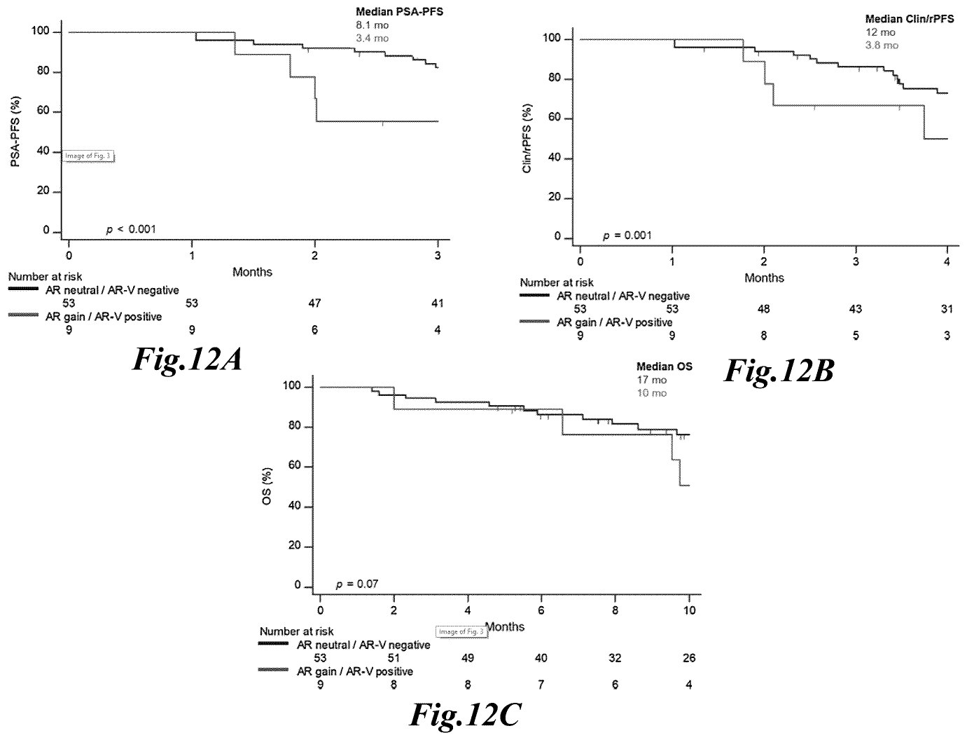

PSA responses were seen in 42/67 (63%) patients, with median PSA-PFS of 7.7 mo. Supplementary Table 8 of Fettke et al. summarizes PSA response rates according to the type of AR aberration. The presence of AR copy number gain was associated with significantly lower PSA response rates (neutral/gain: 28/41 [68%] vs 9/26 [35%], p=0.007), although this difference was observed only in ARPI-treated patients (Supplementary Tables 9 and 10 of Fettke et al.). Supplementary FIG. 2 of Fettke et al. shows the waterfall plot of best PSA response according to AR copy number status. Conversely, the presence of an AR mutation correlated with higher PSA response rates (wild-type/mutated: 26/51 [51%] vs 11/16 [69%], p=0.2); Supplementary FIG. 3 of Fettke et al. shows the waterfall plot of the best PSA response according to AR mutation status. Neither AR-V7 nor AR-V9 transcript expression predicted for a confirmed 50% PSA response. FIGS. 11A-11K demonstrate Kaplan-Meier estimates of PSAPFS according to AR copy number variation, presence of any AR aberration, and total number of AR aberrations, all of which were associated with significantly shorter PSAPFS in univariable (FIG. 11J and multivariable (FIG. 11K) analyses. Furthermore, patients with simultaneous AR gain and AR-V expression (n=9) experienced particularly poor outcomes in univariable analysis (FIGS. 12A-12C and FIG. 11J), median PSA-PFS 8.1 vs 3.4 mo, p<0.001), remaining significant in multivariable analysis (hazard ratio [HR] 4.2, 95% confidence interval [CI] 1.1-15, p=0.03; FIG. 11K). A summary of the univariable associations between AR aberrations and PSA-PFS analyzed by treatment subgroup is shown in Supplementary Tables 11 and 12 of Fettke et al. Importantly, for patients with concurrent AR gain and AR-V expression, PSA-PFS was significantly shorter following both ARPIs (HR 6.7, 95% CI 1.6-29, p=0.009) and chemotherapy (HR 3.9, 95% CI 1.1-15, p=0.04), with median PSA-PFS of 1.8 and 4.3 mo, respectively.

The median clin/rPFS and OS for the overall cohort were 10.4 and 17.1 mo, respectively. Patients with any AR aberration, AR copy number gain, and cumulative AR aberrations experienced significantly shorter clin/rPFS and OS (FIGS. 11A-11K), with the latter two variables remaining significant in multivariable analysis (FIG. 11K). Similar to biochemical outcomes, presence of both AR gain and AR-V expression at baseline predicted poorer clin/rPFS (median 12 vs 3.8 mo, p=0.001) and shorter OS (median 17.1 vs 10.4 mo, p=0.07; FIG. 12A-12C and FIG. 11J. Univariable Cox regression analyses of clin/rPFS and OS for ARPI- and chemotherapy-treated subgroups are shown in Supplementary Tables 11 and 12 of Fettke et al. Notably, for ARPI-treated patients with concurrent AR gain and AR-V expression, clin/rPFS (HR 4.6, 95% CI 1.2-17, p=0.03) and OS (HR 5.1, 95% CI 1.5-17, p=0.008) were significantly shorter, with median clin/rPFS and OS of 2.1 and 11 mo, respectively. Interactions between treatment subgroup and AR aberrations were estimated using a nested Cox proportional hazard regression model for OS (Supplementary Table 13 of Fettke et al.). AR gain, cumulative AR aberrations, and concurrent AR gain plus AR-V expression demonstrated a significant interaction with ARPI therapy.

AR gain was observed to be an independent negative prognostic biomarker for OS and PFS. These findings are consistent with previous reports supporting an association between AR amplification and resistance to ARPI therapy. However, no association was observed between AR point mutations and time-to-event outcomes, even with ligand promiscuity-conferring point mutations L702H, T878A, H875Y, and W742C. Additionally, an association between AR point mutations and increased PSA response rates was observed.

By employing molecular barcoding to uniquely label individual nucleic acid fragments and perform error correction, complemented by deep sequencing of both plasma cfDNA and cfRNA, the successful identification of ultra-low-frequency variants down to 0.1% with minimal input material can be performed.

Using this assay, the understanding of the relationship between AR genomic alterations and aberrant RNA splicing independent of CTC status was observed. Possession of any type of AR aberration and the cumulative number of AR aberrations were independently associated with shorter PFS. In addition, the cumulative number of AR aberrations was independently associated with OS. These findings were not merely a consequence of an increased tumor burden, as the associations remained significant after adjustment for ctDNA fraction. The data illustrate an impact of multianalyte assessment, with cfDNA and cfRNA providing complementary molecular insights into the global influence of the AR in mCRPC.

The impact of AR aberrations was unsurprisingly generally more apparent in the ARPI-treated cohort than in the chemotherapy-treated cohort. A subset of both ARPI- and chemotherapy-treated patients with concurrent AR gain and AR-V expression had particularly poor outcomes (median PSA-PFS 1.8 and 4.3 mo, respectively; HR 6.7 and 3.9, respectively). These results suggest that concurrent DNA and RNA aberrations in the AR may portend to exceedingly aggressive disease biology and inferior outcomes irrespective of treatment. Critically, a shorter OS was observed in patients with both AR gain and AR-V expression, a trend that was able to be observed by using the concurrent DNA and RNA analysis, and may not been readily observable in an assay with only DNA or RNA analysis.

### Example 3: CNV Detection in cfDNA

Samples from multiple subjects with metastatic prostate cancer, including those with metastatic castrate resistant prostate cancer, were obtained. From each patient, a single tube (10 mL) of whole blood was collected in EDTA-containing tubes before undergoing two-step centrifugation to separate plasma and buffy coat. Aliquoted samples were stored at −80° C. for batch processing. cfDNA was extracted from up to 5 mL of plasma using commercially available kits. Large molecular weight DNA fragments were removed, and the remaining nucleic acids were quantified. 5-40 ng of extracted cfDNA or fragmented gDNA was then utilized for library preparation. Preparation of next-generation sequencing (NGS) libraries was performed using DNA end-repair, dA-tailing, adapter ligation and PCR amplification. Amplified DNA libraries subsequently underwent further quality control, before being hybridized overnight to a custom designed targeted panel capturing exonic regions from 90-120 genes. Captured fragments were recovered, washed and further PCR amplified. A final quality control assessment (Bioanalyzer 2100) was performed to confirm the presence of a dominant peak at approximately 300 bp and adequate library quantity (fragments between 200-600 bp>1 nM). Enriched libraries were then sequenced on the Illumina HiSeq XTen.

Simultaneous sequencing of matched white blood cells was also undertaken.

Paired-end reads underwent quality control and sequence alignment using an in-house pipeline that performs barcode checking, adapter trimming, and error correction. Cleaned paired FASTQ files were aligned to the hg19 reference genome using the BWA alignment tool.

Somatic mutation identification was then performed. Consensus BAM files were derived as previously described to minimize sequencing and PCR errors. An in-house pipeline was used to identify candidate somatic variants whilst filtering/removing low-quality variants. A variant was considered a candidate somatic mutation only when all three of the following conditions were met: i) at least four distinct fragments contained the mutation, ii) variant allelic frequency (AF) was at least 0.25%, or 0.1% for hotspot loci (as defined by COSMIC, and iii) variant was absent from public databases of common germline variants (1000 genomes, ExAC, gnomAD and KAVIAR). Candidate somatic mutations were further annotated and filtered to include only missense, nonsense, frameshift, or splice site variants occurring in protein-coding regions. Predicted benign variants (ClinVar) and previously described hematopoietic expansion-related variants were also removed.

Copy number was analyzed for the genes in the targeted panel. Estimation of panel-based copy number variation occurred at the gene level. In-house algorithms calculated the on-target unique fragment coverage based on the consensus BAM file, followed by GC bias correction. Each adjusted coverage profile was self-normalized and then compared against correspondingly adjusted coverages from a group of normal reference samples to estimate the significance of the copy number variation. The minimum gain or loss thresholds were determined based on the CNV change distribution of normal reference samples. Gains or deletions with an absolute z-score >3 and absolute CNV change above minimum gain or loss thresholds were called as true events. To increase the specificity of copy number calls, the pipeline integrates the variant allele frequency information of common single nucleotide polymorphisms (SNPs) located upstream and downstream of the genes in panel. A CNV call algorithm was used to detect gene level copy number gains and losses. Additional, orthogonal validation with low-pass whole genome sequencing (LP-WGS) was performed with an overall average coverage of 2× on patient samples with sufficient additional cfDNA following targeted panel sequencing (n=46). The ichorCNA tool algorithm6 was applied to GC and mappability-normalized reads to estimate plasma copy number variations using a hidden Markov model (HMM) with 1-megabase resolution. Multiple initial normal cell probabilities were tried during the Expectation-Maximization (EM) initialization step of ichorCNA software to find the optimized LP-WGS copy number status estimation. To call a copy number gain or loss, the copy number change should pass minimum threshold: larger than 5% change for autosomal genes and 10% for genes on X chromosome.

Kaplan-Meier survival estimates (log-rank test) and multivariable Cox regression models were used to assess the association between PI3K/Akt pathway aberrations and clinical outcomes including progression-free survival (PFS; time from treatment commencement to first of confirmed PSA progression, clinical or radiographic progression, or death from prostate cancer) and overall survival (OS; time from treatment commencement until death from any cause). Where an event had not occurred at time of data analysis, survival outcomes were right censored at the date of last patient contact.

The assay was performed using a custom targeted panel-based approach, in combination with a software analysis algorithm. Hybrid capture probes targeting single nucleotide polymorphisms (SNP) in the introns both upstream and downstream of relevant genes were employed to capture additional copy number information. By integrating both coverage and SNP allele frequency change information, the assay can detect CNV events with high sensitivity and specificity.

PTEN loss was observed in 37% (85/231) of patients. In a subset of patients with additional plasma available (n=46, 59%), LP-WGS confirmed targeted panel-detected PTEN loss in 81% (25/31) of patients (eTable X of the Supplement). Close examination of the LP-WGS profile surrounding the PTEN locus in the six patients with discordant results showed either the presence of focal copy number loss (n=4), or a transition boundary between adjacent copy number loss and copy number neutral segments (n=2), suggesting enhanced capability of the assay for detecting focal and global CNVs in blood.

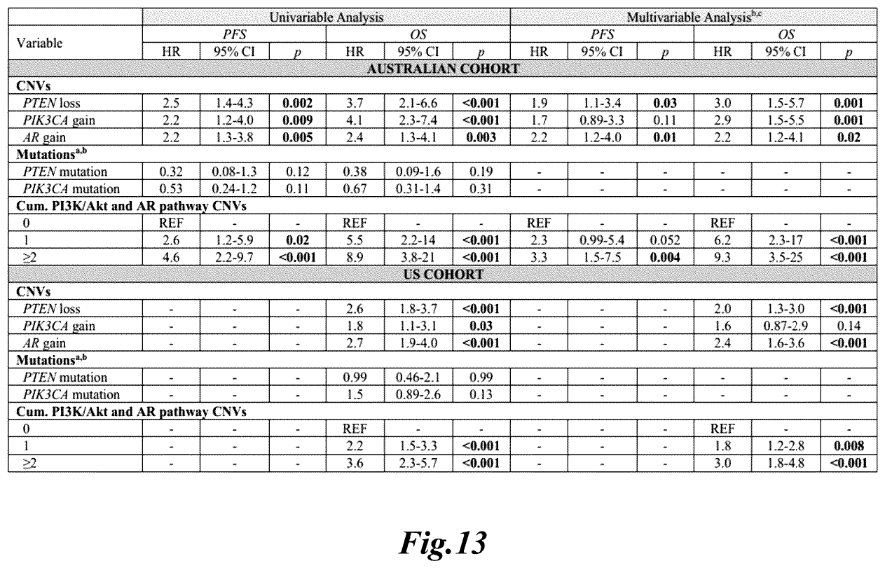

PTEN loss was strongly associated with PFS and OS, remaining significant in multivariable analysis when adjusting for baseline clinicopathologic prognostic factors as well as ctDNA fraction (FIG. 13). A similar association was confirmed in the US cohort with respect to OS (FIG. 13). Of note, in the 146 PTEN-copy number neutral patients in this study, 31 (21%) patients exhibited alternate PI3K/Akt pathway aberrations, most commonly PIK3CA mutation/gain (n=21, 68%) and PTEN mutation (n=7, 23%). In addition, none of the patients with mutations in AKT1 (n=5) or mTOR (n=2) across either cohort harbored concurrent PTEN loss and/or mutation.

PIK3CA gain was observed in 17% (39/231) of patients. In a subset of Australian cohort patients with additional plasma available, LP-WGS confirmed targeted panel-detected PIK3CA gain in 94% (16/17) of patients. PIK3CA gain was independently associated with poor survival outcomes in the Australian cohort, but not the US cohort (FIG. 13).

In the Australian cohort, somatic mutations were most frequently observed in PIK3CA (13/78, 17%). PTEN mutations were uncommon at 6% (5/78), with AKT1 and mTOR mutations rare at a single case each. In the US cohort, PIK3CA mutations were again the most common, albeit at a lower prevalence than the Australian cohort at 10% (15/153). PTEN, AKT1 and mTOR mutation were observed in <5% of patients. Given the low frequency of certain PI3K/Akt pathway mutations (e.g. AKT1, mTOR) in both cohorts, correlation with clinical outcomes was restricted to genes mutated in at least five patient samples. In contrast to CNVs, mutations in PI3K/Akt pathway genes did not significantly correlate with clinical outcomes (FIG. 13). A full list of PI3K/Akt pathway CNVs and mutations for the Australian and US cohort can be found in eTable 6 and eTable 7 of the Supplement, respectively.

AR gain was present in 51% (40/78) of patients in the Australian cohort, and 37% (56/78) of patients in the US cohort, and was associated with shorter PFS and OS in univariable and multivariable analysis in both cohorts (FIG. 13).

Considering PTEN loss, PIK3CA gain and AR gain collectively across both cohorts, 0/1/≥2 CNVs were observed in 102 (44%), 62 (27%) and 67 (29%) samples. Cumulative CNVs in the PI3K/Akt and AR pathways were significantly associated with OS in both the Australian (median OS for 0 vs ≥2 CNVs: 33.5 mo vs 9.7 mo, p<0.001) and US cohort (median OS for 0 vs ≥2 CNVs: 35.5 mo vs 9.2 mo, p<0.001. Critically, this relationship persisted when multivariable analysis was performed on each cohort separately (FIG. 13). Analysis of outcomes by treatment subgroup in the Australian cohort demonstrated findings were most apparent as expected in ARPI-treated patients.

While isolated PTEN loss was associated with less favorable outcomes (median OS 11 mo vs 21 mo, HR 1.6, 95%1.0-2.7), the negative impact of PIK3CA gain was most apparent with combined PTEN/PIK3CA CNV (median OS 7.2 mo vs 21 mo, HR 2.3, 95% CI 1.2-4.4) and PTEN/PIK3CA/AR CNV (median OS 8.5 mo vs 21 mo, HR 3.2, 95% CI 2.0-5.0) (Table 7). To determine if treatment exposure impacted the prevalence of dual PI3K/Akt pathway CNV activation, rates of prior systemic therapy in patients with PTEN loss without PIK3CA gain (n=50) were compared versus patients with PTEN loss and PIK3CA gain (n=35). There was no difference with respect to prior ARPI therapy (yes vs no: 56% vs 38%, p=0.3), taxane chemotherapy (39% vs 44%, p=1.0), or any systemic therapy (42% vs 38%, p=0.9).

TABLE 7
Hazard ratio for overall survival attributable
to PI3K/AR pathway CNVs

| Variable | N(%) | HR | 95% CI |
|---|---|---|---|
| No PI3k/AR pathway CNV | 102(44) | 0.32 | 0.23-0.42 |
| AR gain alone | 40(17) | 1.5 | 1.0-2.1 |
| PIK3CA gain alone | 2(1) | 0.6 | 0.15-2.4 |
| PTEN los alone | 20(9) | 1.6 | 1.0-2.7 |
| AR gain + PTEN los | 30(13) | 2 | 1.3-3.0 |
| AR gain + PIK3CA gain | 2(1) | 2.5 | 0.62-10 |
| PIK3CA gain + PTEN loss | 11(5) | 2.3 | 1.2-4.4 |
| AR gain + PTEN loss + | 24 (10) | 3.2 | 2.0-5.0 |
| PIK3CA gain | | | |

### Example 4: Use of Algorithm for Cancer Detection and Prognosis

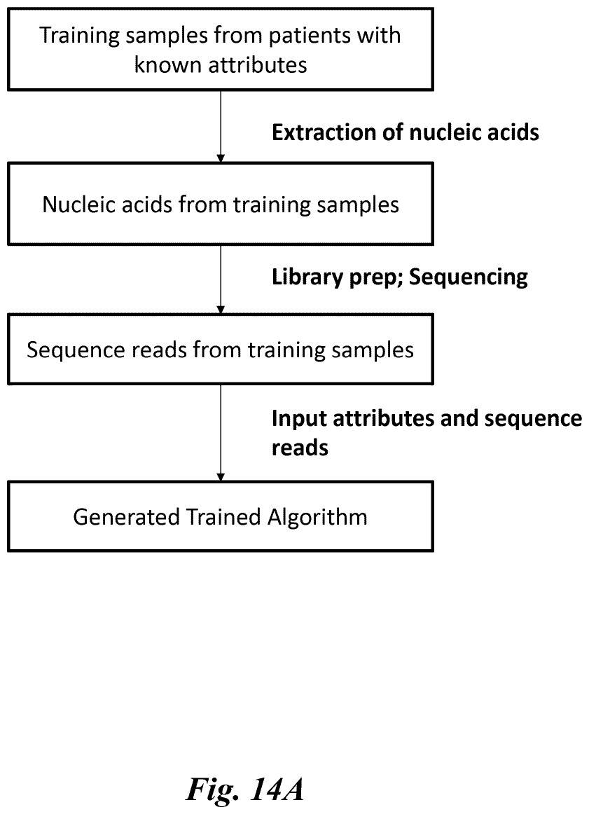

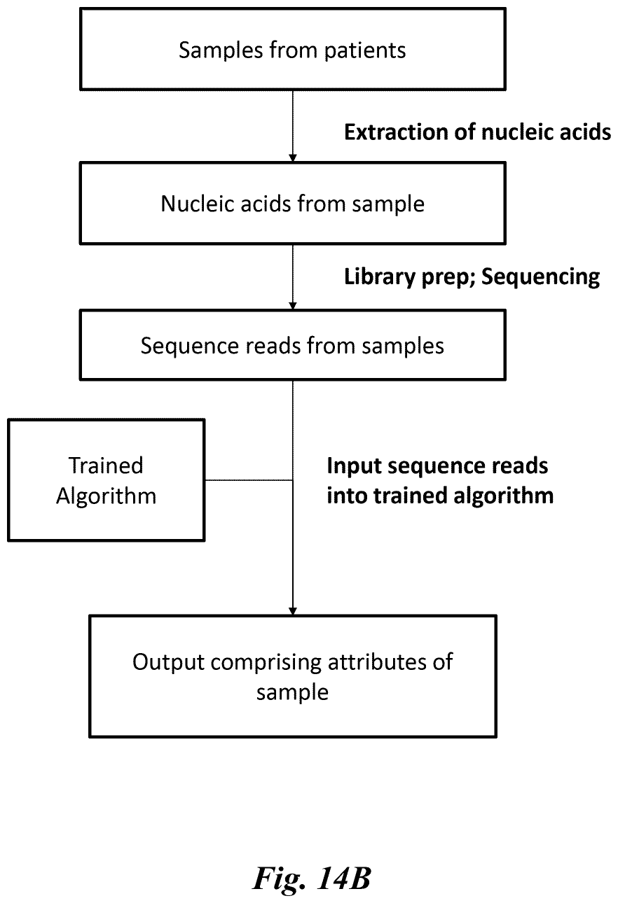

As described in Examples 1-3, sample from patients are obtained. Nucleic acids corresponding DNA and RNA, such as germline DNA, cfDNA, and cfRNA are extracted, processed and sequenced to generate sequence reads derived from the nucleic acids. Based on the attributes of the subject the samples are derived from (e.g. normal, has mCRPC), the samples may be inputted into an algorithm (e.g. trained algorithm) and allowed to process the sequencing reads and sample attributes. The algorithm may be a machine learning algorithm and process the sequences reads and sample attributes such to identify correlations, clusters, trees, or other associative measures and be allowed to identify markers that are associated or indicative of a sample attribute. This algorithm may be trained by these samples to determine if given sample is indicative of a attribute of the sample or subject from which the sample is derived. FIG. 14A shows a schematic of this workflow.

A sample from a subject that is suspected of cancer, or who has had treatment for a cancer is obtained. The attributes of the sample may be partially unknown, for example, the effectiveness of the treatment may not be observed, or the type of cancer may not be understood. The nucleic acids of the sample are extracted as described elsewhere herein, and the nucleic acids are subjected to reactions and sequencing. The sequencing reads are then processed using the algorithm that has been trained on a plurality of training samples. The algorithm processes the sequence reads and identifies biomarkers of interest. Upon processing, the algorithm outputs a report that may have at least one of the following outputs relating to attributes of the sample, for example, if a cancer is still observed, the type of cancer, if the cancer contains biomarkers indicative of drug resistance, as well as differences between this sample and another sample from the subject (in the case that prior sample has been obtained). The output may also contain a probability or likelihood metric, or a confidence metric for a given attribute. FIG. 14B shows a schematic of this workflow.

### Example 5: Circulating Cell-Free DNA-Based Detection of Tumor Suppressor Gene Copy Number Loss

Paired tumor tissue-plasma samples collected from 15 metastatic castration-resistant prostate cancer patients were analyzed by a PredicineCARE assay (a hybrid capture based NGS-targeted liquid biopsy assay), low-pass whole genome sequencing (LP-WGS), and immunohistochemical (IHC) assays. Current liquid biopsy assays may lack sufficient sensitivity to detect copy number loss, which limits the interrogation of critical tumor suppressor gene deletions during cancer progression and treatment. A liquid biopsy assay described in this example showed improved sensitivity for detection of copy number loss in blood samples with low levels of circulating tumor DNA, and demonstrate its utility by profiling PTEN, RB1, and TP53 genetic loss in metastatic prostate cancer patients.

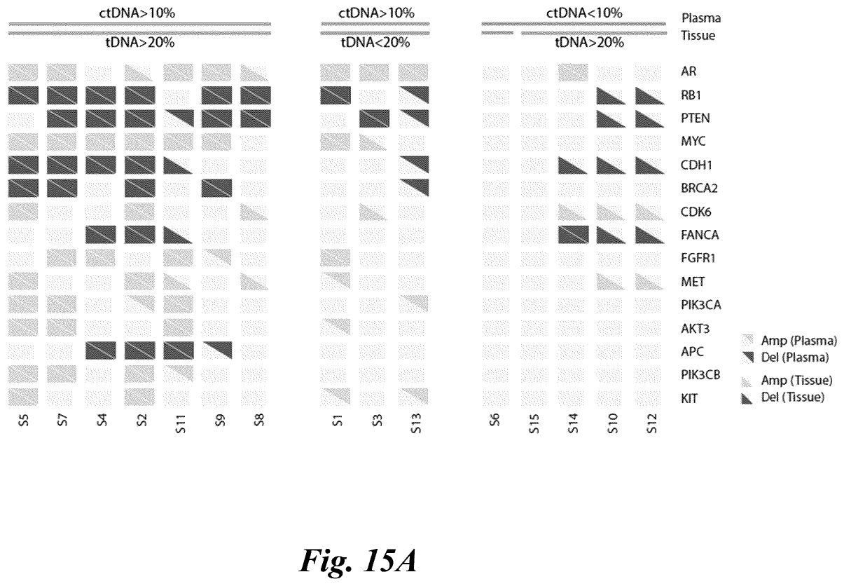

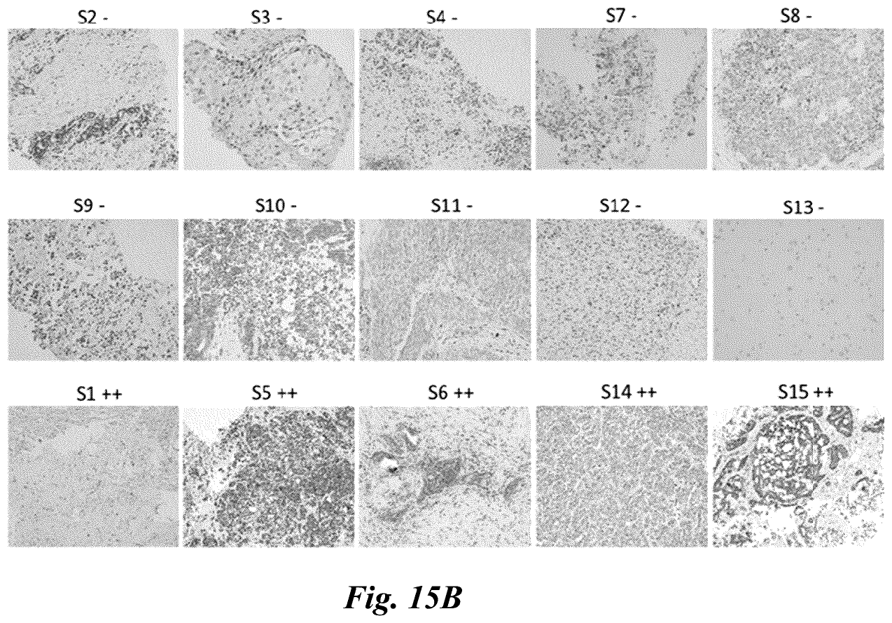

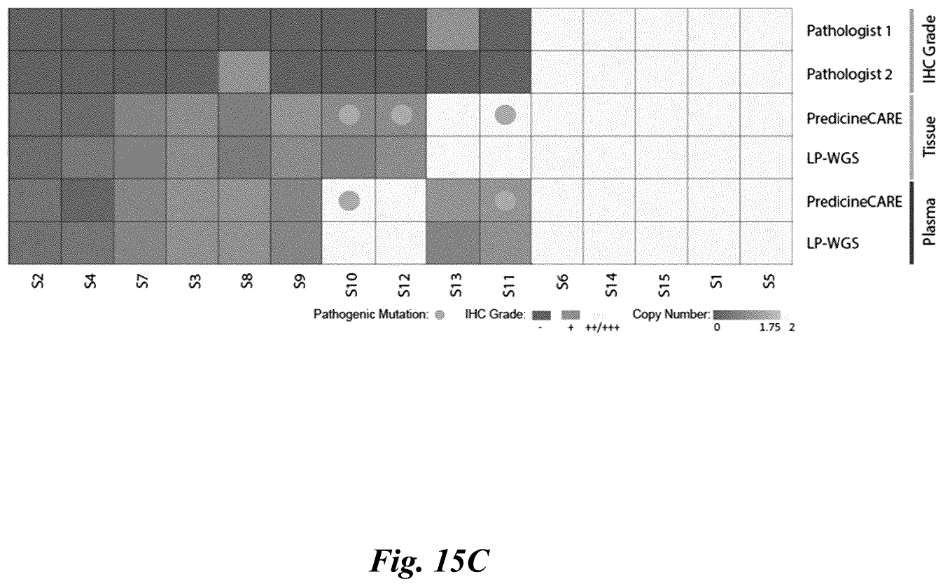

FIG. 15A shows the landscape of gene copy number variations, including amplification and deletion events detected by the PredicineCARE assay in tissue and plasma samples. Genes that were altered in >10% of the samples are shown. Samples are grouped according to circulating tumor DNA (ctDNA) and tissue tumor DNA (tDNA) fractions. ctDNA fractions were estimated by LP-WGS and mutation allele frequency reported by PredicineCARE assay. Tissue tumor DNA (DNA) fraction levels were estimated by pathological reviews. “Amp” indicates a gene amplification (copy number gain), whereas “Del” indicates a gene deletion (copy number loss). FIG. 15B shows images relating to PTEN expression in 15 prostate cancer tissues as detected by immunohistochemistry (×20). Pathology score is defined as the product of Staining intensity score (0-3) and Stained area score (0-3). If the score ≤1, the sample is determined as negative (−). If the score>1, the result is positive (Score 1-3, grade+; Score 4-6, grade++; Score 7-9, grade+++). Two pathologists reviewed the slides independently and average score was considered as the final score for a given case. FIG. 15C shows a chart demonstrating the agreement of PTEN loss between tissue and blood-based detection for the 15 pair of samples. “IHC grade” shows PTEN protein expression by immunohistochemical staining. PTEN gene copy loss at DNA level was evaluated in tissue and plasma samples using the PredicineCARE assay and low pass whole genome sequencing assay (LP-WGS).

Estimation of panel-based copy number variation occurred at the gene level. In-house algorithms were used to calculate the on-target unique fragment coverage based on the consensus BAM file, followed by GC bias correction. Each adjusted coverage profile was self-normalized and then compared against correspondingly adjusted coverages from a group of normal reference samples to estimate the significance of the copy number variation. The minimum gain or loss thresholds were determined based on the CNV change distribution of normal reference samples. Gains or deletions with an absolute z-score >3 (the inverse of the cumulative distribution function of a Gaussian distribution at 99.59% confidence level) and absolute CNV change above minimum gain or loss thresholds were called as true events.

To increase the specificity of copy number calls, the pipeline integrates the variant allele frequency information of common SNPs located up to 1 Mb upstream and downstream of the genes in panel. If there is only one SNP allele with altered MAF or with a significantly different copy number to the other allele, then the allele variant frequency of the heterozygous SNPs will shift away from the expected 0.5. We define the average variant frequency deviation from 0.5, AF_{shift}, as

$$Δ {F}_{shift} = \frac{1}{N} {\sum }_{i = 1}^{N} \vert {AF}_{i} - 0.5 \vert$$

where AFᵢ is the variant frequency of the SNPᵢ, N is the number of heterozygous SNPs located in the gene region (including up to 1 Mb upstream and downstream of the gene body). The pipeline considers the change of AF_{shift} is significant if AF_{shift}>3*SD_{AF} where SD_{AF} is the standard deviation of SNP variant frequency AF;, and at least 3 supporting heterozygous SNPs (N≥3) are required to call a significant AF_{shift}. The CNV pipeline detects a gene with CNV changes if it satisfies both copy number changes and AF_{shift} thresholds. For genes without heterozygous SNP support, or having heterozygous SNP coverage but lacking of SNP AF_{shift} support, a more stringent gene copy number change threshold (1.5× of minimum copy number change threshold) is applied to make a confident CNV call.

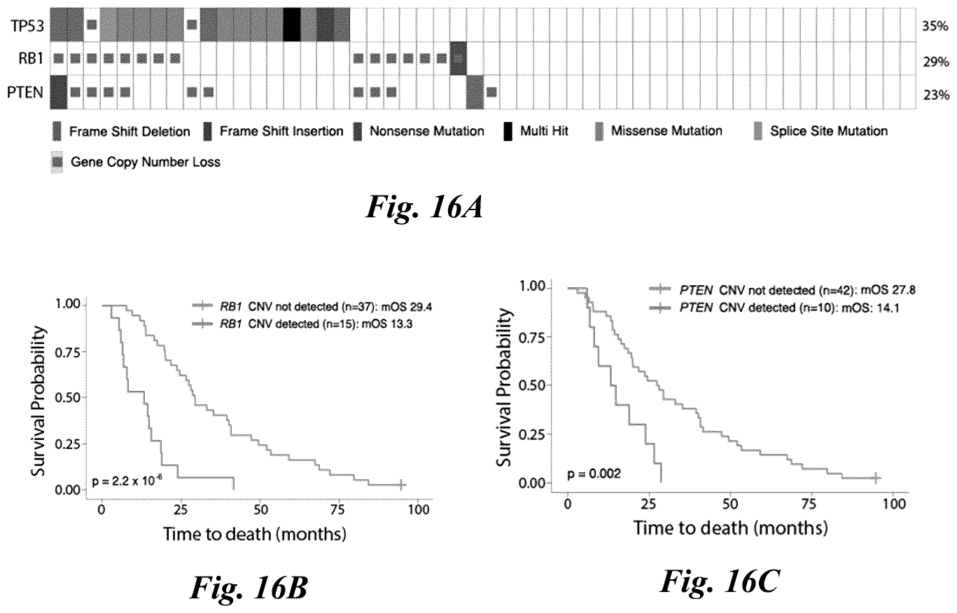

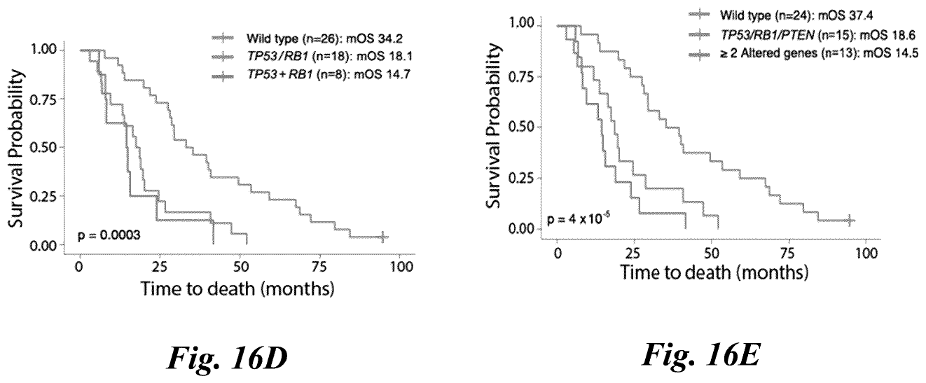

Additionally, 52 mCRPC plasma samples were analyzed for genomic alterations of TP53, RB1, and PTEN as well as overall survival (OS). FIG. 16A shows a genomic landscape of PTEN, RB1 and TP53 in 52 mCRPC patients, including copy number variations (CNVs), single nucleotide variations (SNVs) and short insertions/deletions, reported by the cfDNA-based PredicineCARE assay. Blood samples were collected from patients before chemotherapy treatment. The percentage of samples having aberrations (SNV+CNV) in each gene is listed to the right of the heatmap. FIGS. 16B-16E shows the Kaplan-Meier analysis of overall survival (OS) according to PTEN, RB1 and TP53 loss status. OS is plotted for different patient groups classified according to (FIG. 16B) RB1 copy loss status, (FIG. 16C) PTEN copy loss status, (FIG. 16D) Alterations (SNV and/or CNV) in TP53 and/or RB1, and (FIG. 16C) Alterations (SNV and/or CNV) in one vs. more than one of the PTEN, RB1 and TP53 genes. “mOS” is the median overall survival. “TP53/RB1” indicates that either TP53 or RB1 was aberrant. “TP53+RB1” indicates that both TP53 and RB1 were aberrant. “TP53/RB1/PTEN” means that any one of TP53, RB1 or PTEN was aberrant. “>2 Altered Genes” indicates instances where at least two genes among the TP53, RB1 or PTEN triad harbored genomic aberrations.

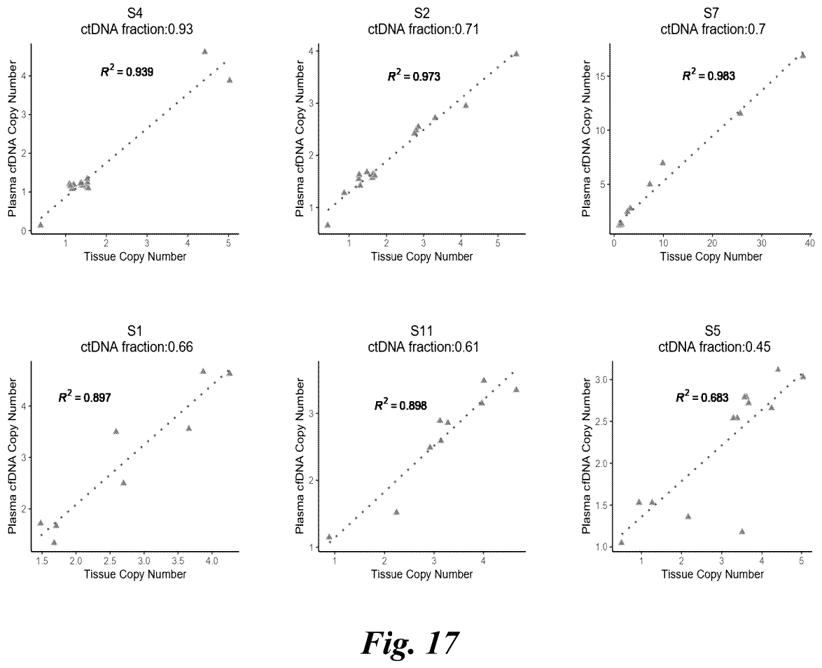

FIG. 17 shows correlation between copy numbers estimated from liquid and tissue biopsies for genes with both tissue and liquid biopsy CNV calls in the paired samples. Each gene is represented as a single data point. The same hybrid-capture based panel assay (PredicineCARE) was used for liquid and tissue biopsies. The dashed line represents the fitted linear regression equation.

### Example 6: Urinary Molecular Pathology for Patients with Newly Diagnosed Urothelial Bladder Cancer

Next-generation sequencing (NGS)-based profiling of both urinary tumor DNA (utDNA) and circulating tumor DNA (ctDNA) shows promise for noninvasive detection and surveillance of urothelial bladder cancer (UBC). In the present example, the concordance between tumor DNA (tDNA) profiling and utDNA or ctDNA assays was evaluated using a UBC patient cohort from the intended-use population.

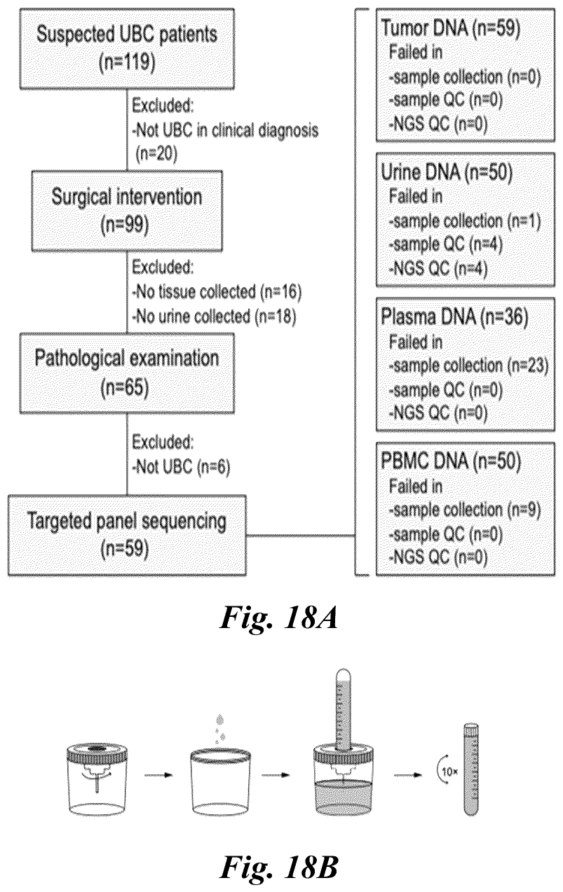

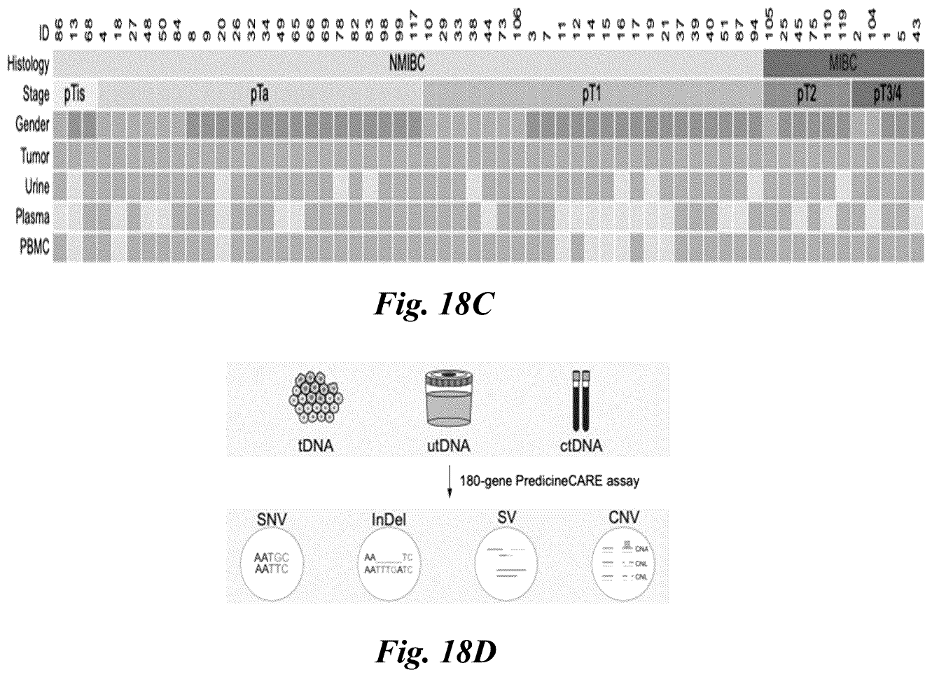

Fifty-nine cases with pathologically confirmed disease and matching tissue/urine pairs were prospectively enrolled. Baseline peripheral blood mononuclear cell and plasma specimens were collected during clinic visits. The PredicineCARE NGS assay was applied for ultra-deep targeted sequencing and somatic alteration identification in tDNA, utDNA, and ctDNA. Diverse quantitative metrics including CCF (cancer cell fraction), VAF (variant allele frequency) and TMB (tumor mutation burden) were invariably concordant between tDNA and utDNA, but not ctDNA. The mutational landscape captured by tDNA or utDNA were highly similar, whereas a considerable proportion of ctDNA aberrations stemmed from clonal hematopoiesis. Using tDNA-informed somatic events as reference, utDNA assays achieved a specificity of 99.3%, a sensitivity of 86.7%, a positive predictive value of 67.2%, a negative predictive value of 99.8%, and a diagnostic accuracy of 99.1%. Higher preoperative utDNA or tDNA abundance correlated with worse relapse-free survival. Actionable variants including FGFR3 alteration and ERBB2 amplification were identified in utDNA. FIGS. 18A-D show the patient cohort and study design.

FIG. 18A shows flow chart depicting patient selection and sample sequencing. The number of enrolled patients or analyzed samples was shown for each stage of the study. FIG. 18B shows illustration of a customized device for self-support urine sample collection. First morning urine was voided to the storage cup, inhaled into vacuum-based collection tubes, and mixed with prefilled preservation solution by inverting the tube 10 times. FIG. 18C shows graphical overview of clinicopathological parameters for UBC patients and summarized status of sample NGS analyses. Pink in Gender: female; Grey in Gender: male; Blue: data available; Light grey: data not available. FIG. 18D shows a schematic for the Precidine Care assay. The PredicineCARE assay was used to call SNV, InDel, SV and CNV from tDNA, utDNA and ctDNA. UBC=urothelial bladder cancer; PBMC=peripheral blood mononuclear cells; NGS=next-generation sequencing; QC=quality control; tDNA=tumor DNA; utDNA=urinary tumor DNA; ctDNA=circulating tumor DNA; SNV=single nucleotide variant; InDel=insertion and deletion; SV=structural variation; CNV=copy number variation.

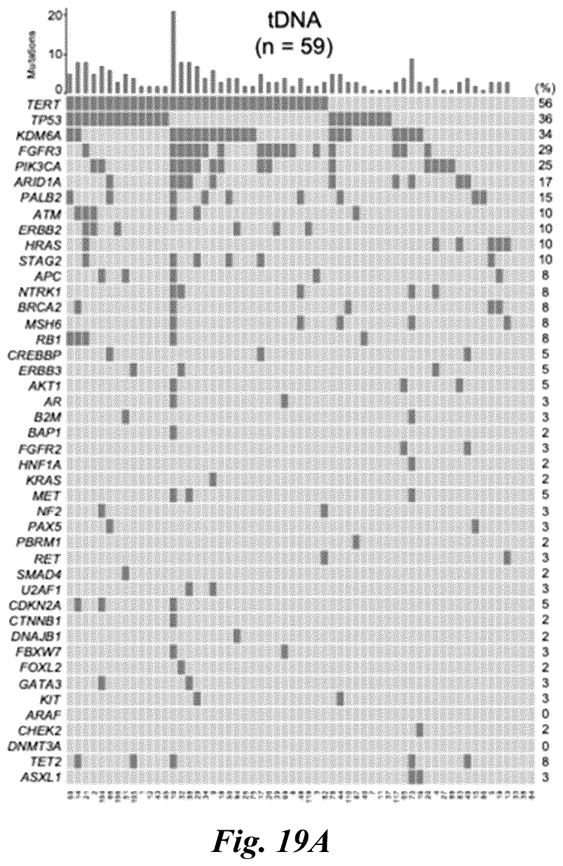

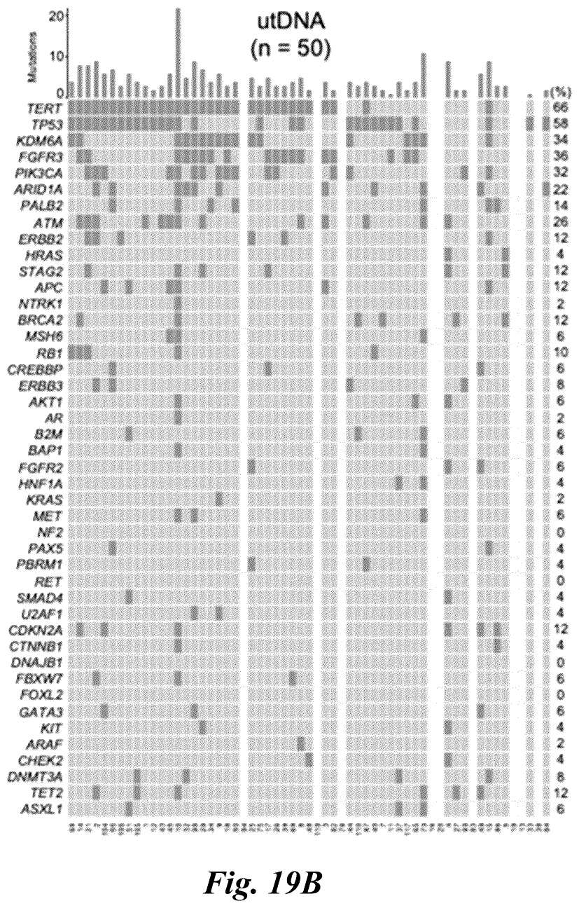

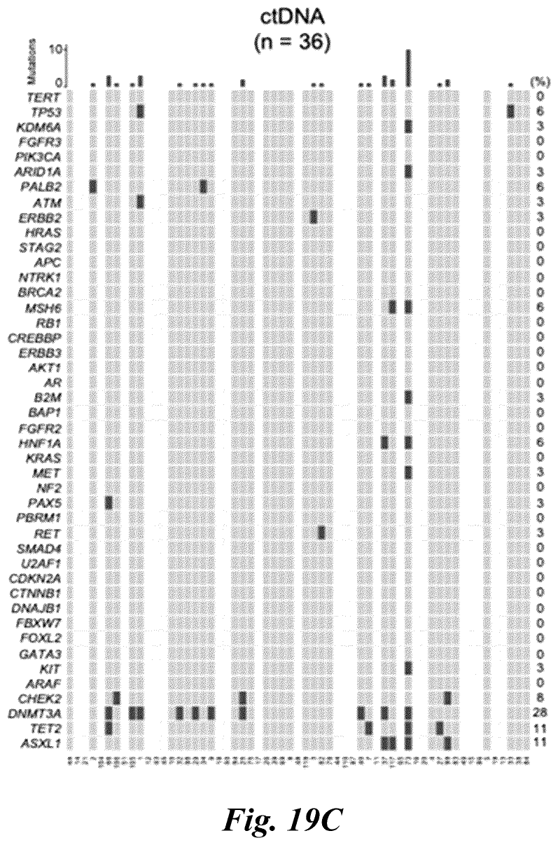

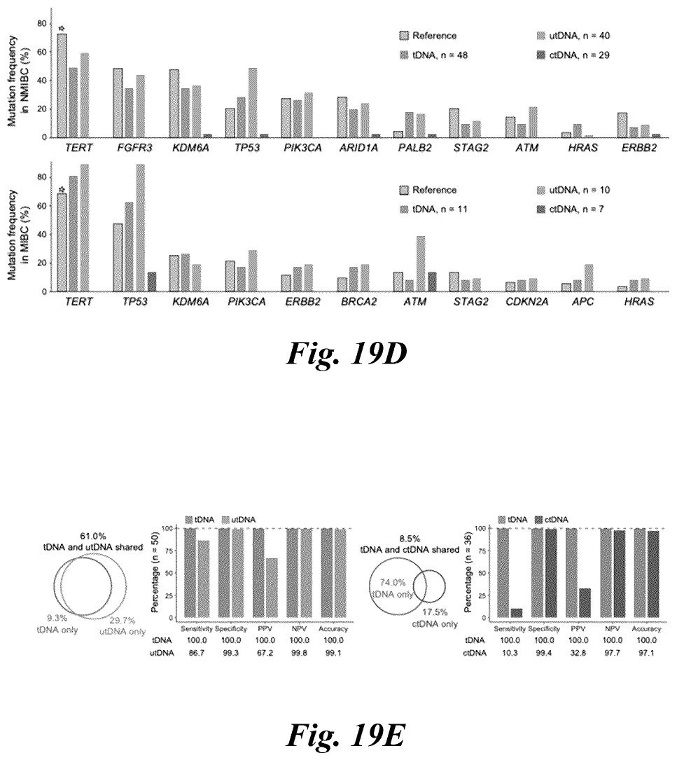

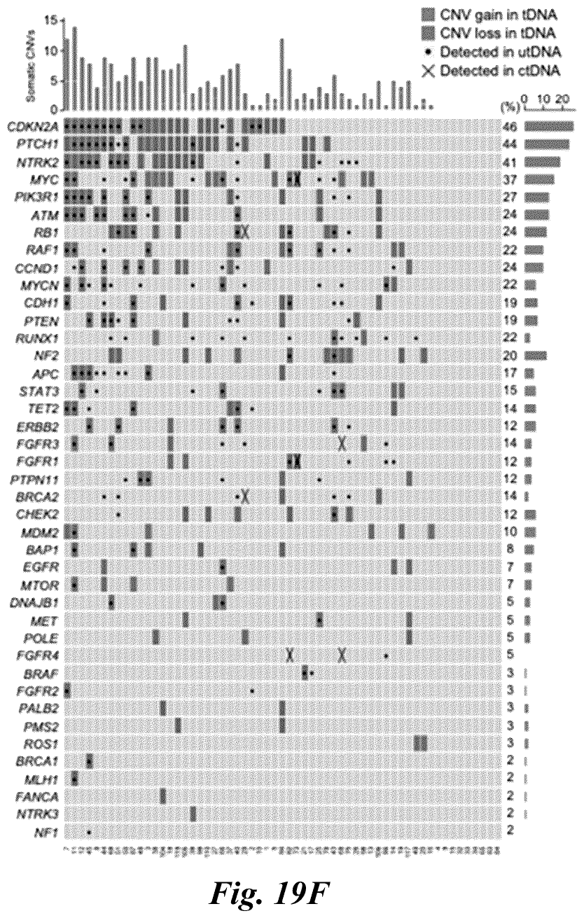

FIGS. 19A-19F show charts relating to somatic aberrations detected in tumor tissues and liquid biopsies. FIGS. 19A-19C show Oncoprint chart for the mutational landscape of tDNA (FIG. 19A), utDNA (FIG. 19B) and ctDNA (FIG. 19A). A total of 59 patients were tested by PredicineCARE assay and mutation frequencies of each gene were shown on the right. FIG. 19D shows the comparison of mutation frequencies in tDNA, utDNA and ctDNA from NMIBC and MIBC patients. The grey bar referred to the mutation frequencies reported by Memorial Sloan Kettering Cancer Center (NMIBC), The Cancer Genome Atlas (MIBC), or the German Cancer Research Center for TERT promoter mutations (asterisk). FIG. 19E shows Venn plots showed the proportion of overlapping variants called by tDNA and cfDNA sequencing. Bar plots showed the variant-level sensitivity, specificity, PPV, NPV, and accuracy of liquid biopsies using tDNA-informed results as the ground truth. FIG. 19F shows a heatmap for inferred CNV in tDNA, utDNA and ctDNA. Patients were arranged along the x-axis. Genes with CNV gain or loss were listed on the left. The corresponding CNV frequencies in tDNA were shown on the right. NMIBC=non-muscle invasive bladder cancer; MIBC=muscle invasive bladder cancer; PPV=positive predictive value; NPV=negative predictive value; CNV=copy number variation.

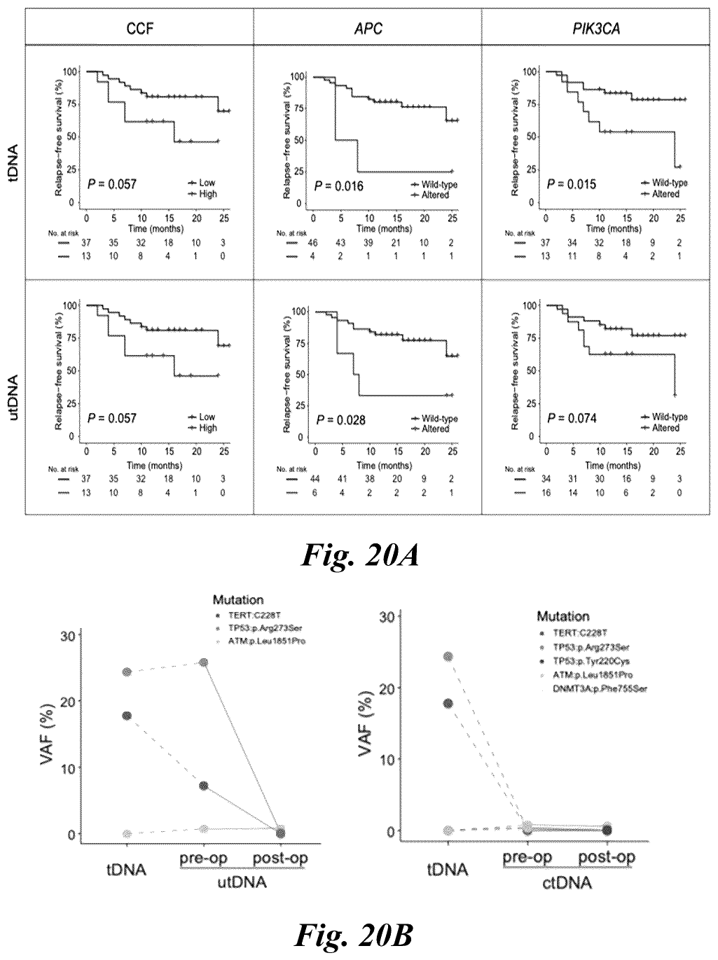

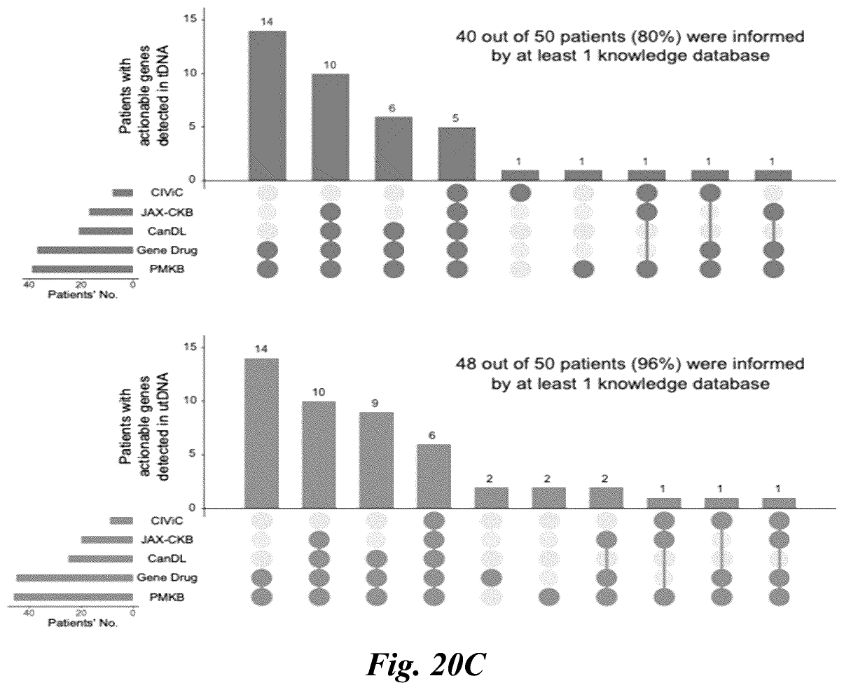

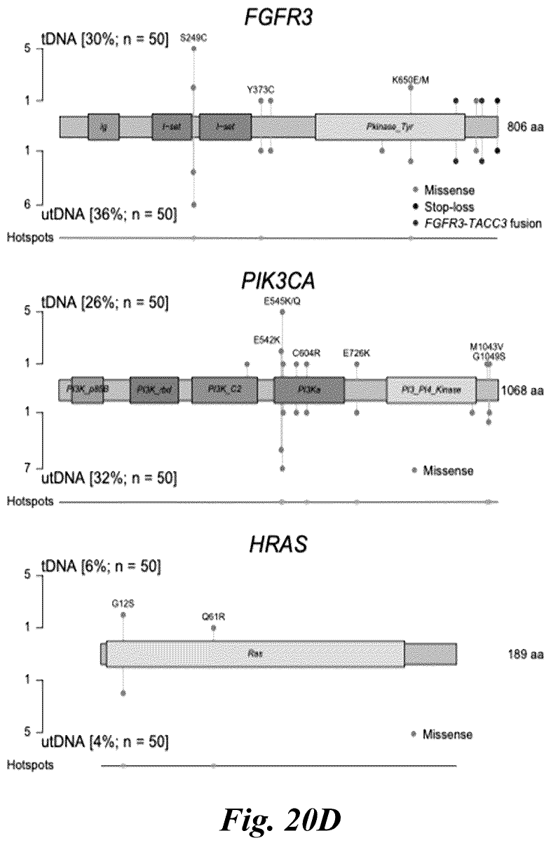

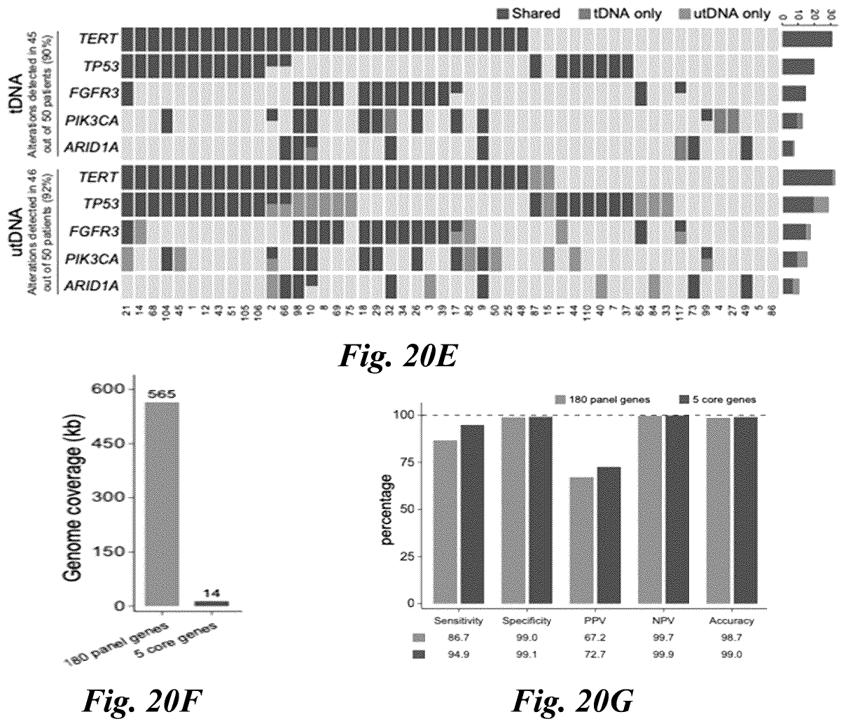

FIGS. 20A-20F show the clinical implications of utDNA analysis in UBC. FIG. 20A Kaplan-Meier analysis of relapse-free survival in 50 patients according to the level of CCF (left), alteration of APC (middle), and alteration of PIK3CA (right) from tDNA (upper) or utDNA (lower) testing. P-values were based on log-rank tests. FIG. 20B shows the dynamic perioperative changes of mutations in one patient with paired pre- and post-operation cfDNA available. The VAF of TERT and TP53 variants only decreased in utDNA after curative-intent surgery. FIG. 20C shows UpSet plots showed UBC patients with actionable genes identified by tDNA (upper) and utDNA (lower) testing. The five cancer variant knowledgebases used in this study were shown on the left. FIG. 20D shows a lollipop view of the mutation pattern for three actionable genes in tDNA and utDNA. Recurrent hotspot mutations were marked according to the cBioPortal database (www.cbioportal.org/visualize). FIG. 20E shows somatic alterations of 5-core genes detected in tDNA (upper) and utDNA (lower). 50 patients were arranged along the x-axis, and 5 genes were listed on the y-axis. FIG. 20F shows a bar plot showed genome coverage of the original PredicineCARE panel versus the simplified 5-gene panel. FIG. 20G shows bar plots indicated high concordance of utDNA testing using PredicineCARE or 5-gene panel. CIVIC=Clinical Interpretation of Variants in Cancer; JAX-CKB=JAX Clinical Knowledgebase; CanDL=Cancer Driver Log; Gene Drug=Gene Drug Knowledge Database; PMKB=Precision Medicine Knowledgebase

### Example 7: Blood Tumor Mutational Burden and Blood Copy Number Burden by Genome-Wide Circulating Tumor DNA Assessment Predict Outcome and Resistance in Hormone-Receptor Positive, HER2 Negative Metastatic Breast Cancer Patients Treated with CDK4/6 Inhibitor

CDK4/6 inhibitors combined with endocrine therapy improve survival for HR+, HER2-MBC. However, biomarkers to predict efficacy and resistance are needed. Using comprehensive next-generation sequencing (NGS)-based liquid biopsy assessment of ctDNA mutation and copy number burden as described in this example identified novel prognostic and predictive biomarkers. PredicineWES+, an assay that combines whole exome sequencing with deep coverage of 600 cancer genes targeted by the PredicineATLAS panel, was used to generate genomic profiles of somatic single nucleotide variation (SNV), indels and copy number variation (CNV), and determine blood tumor mutation burden (bTMB) scores reflecting the number of mutations per megabase of DNA. LP-WGS was used to generate blood copy number burden (bCNB) scores representing a comprehensive measure of copy number variation, including amplifications and deletions across all chromosome arms, and tumor burden/shedding in the blood.

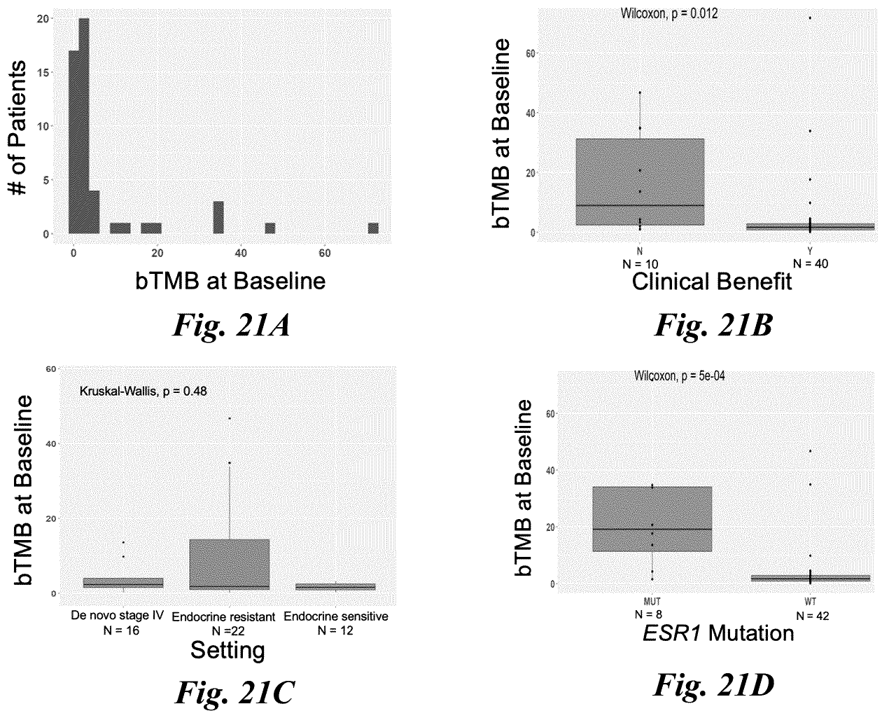

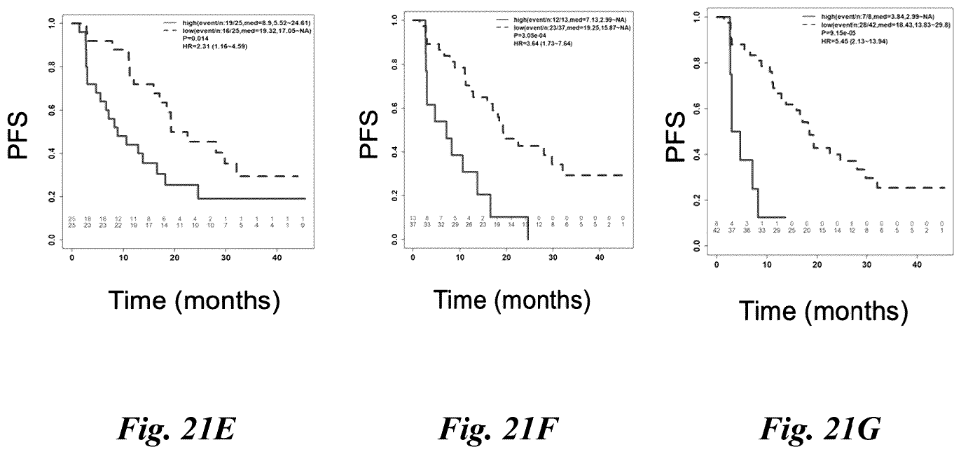

FIGS. 21A-21G show charts relating bTMB and patient outcomes. High bTMB and association with poor patient outcomes. FIG. 21A shows the distribution of bTMB scores across 50 baseline patient samples sequenced by PredicineWES+. High bTMB scores were significantly associated with lack of clinical benefit (CB) defined as PD within 6 months, as demonstrated in FIG. 21B, and the presence of ESR1 mutations at baseline, shown in FIG. 21C. High bTMB scores were more common in the (FIG. 21D) endocrine resistant as compared to the endocrine sensitive cohort (per ESMO 2020 guidelines) and associated with significantly shorter PFS based on the following cutoffs for bTMB: median (FIG. 21E), third quartile (FIG. 21F), and bTMB score of 10 (FIG. 21G).

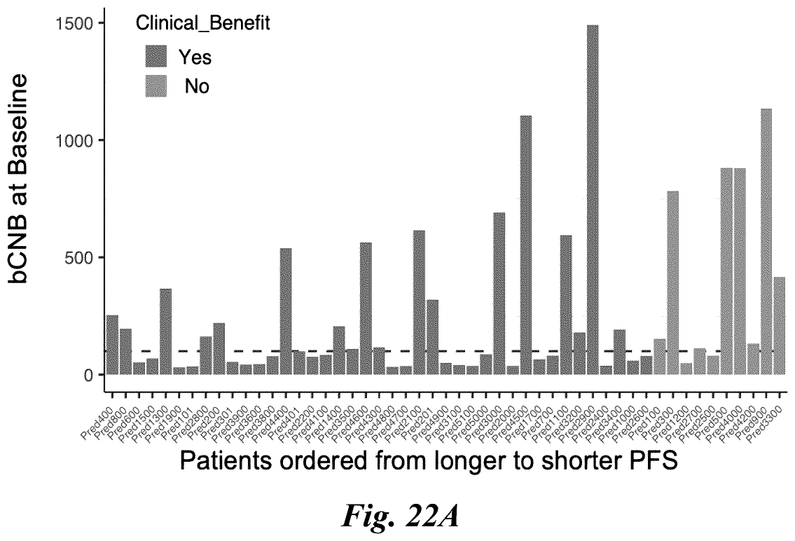

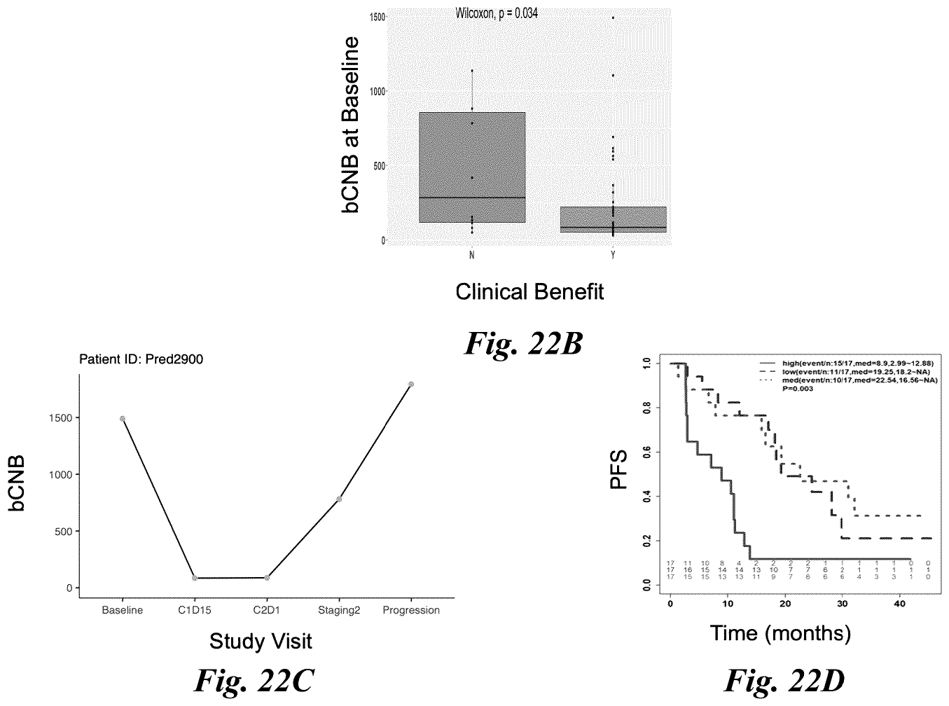

FIGS. 22A-22D demonstrate dynamic changes in bCNB predict patient outcomes and precede radiographic response and clinical progression. FIG. 22A shows the bCNB scores across 51 patients at baseline. FIG. 22B shows a chart related bCNB and clinical benefit (CB) High bCNB scores (>100) were significantly associated with lack of CB. FIG. 22C shows a serial analysis of bCNB during treatment. This shows a revealed decreases at CID15/C2D1, followed by increases that preceded imaging detection of progressive disease in 9/18 of patients for whom staging blood samples were analyzed. FIG. 22D shows that high CID15/BL bCNB ratios were significantly associated with shorter PFS.

Using two comprehensive NGS platforms, PredicineWES+ and LP-WGS, to profile hormone positive HER2-metastatic breast cancer patients receiving ET+CDK4/6 inhibitors, it was demonstrated that: (1) high bTMB and bCNB scores identify a subset of patients who do not receive clinical benefit, (2) dynamic changes in bCNB scores predict outcomes and precede clinical progression.

PredicineWES+ allows for deriving TMB to plasma, detects additional prognostic biomarkers at baseline and reveals novel alterations at progression that may underly resistance.

### Example 8: Whole Exome and Whole Genome Methylation Sequencing of Low Input cfDNA to Implement Precision Medicine in Metastatic Castration Resistant Prostate Cancer

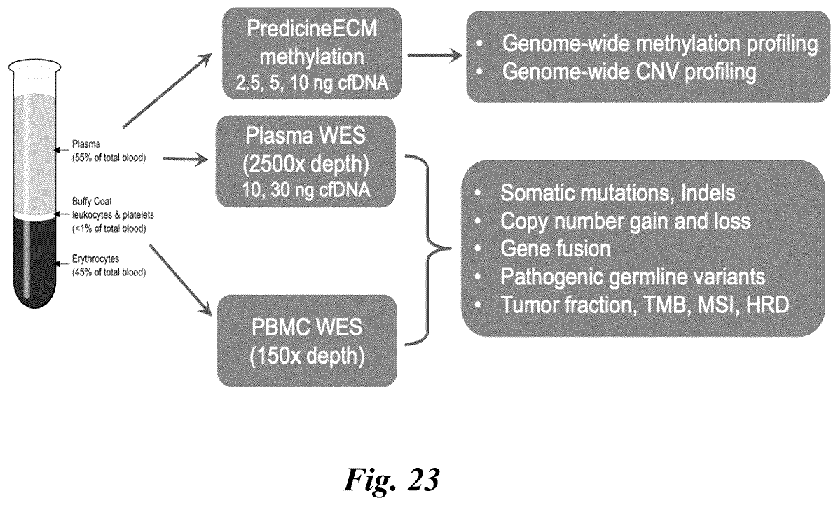

Liquid biopsy has become increasingly important in cancer diagnosis, personalized medicine, and disease progression monitoring. Conventional liquid biopsy relies on targeted cancer gene panels which often contain fewer than 500 genes. Despite the revolutionary impact it has brought to cancer research and patient care, targeted gene panels may miss key novel mutations involved in cancer development and drug response, and other novel genomic and/or epigenomic alternations underlining cancer development, such as whole genome structural or DNA methylation changes. Additionally, current commonly used methodologies require relatively large amounts of cell-free DNA (cfDNA) input material. To this end, this example demonstrates the development of a multiomics analysis platform for low-input cfDNA (10-30 ng) that integrates whole exome mutation profiling (plasma WES) and whole genome methylation profiling (PredicineECM) and applied this method to investigate prostate cancer patient samples. FIG. 23 shows a schematic of the general workflow of the plasma WES and methylation profiling.

Plasma WES profiling of 30 or 10 ng cfDNA detected variants with mutation allele frequency (MAF) of 1% and above in the entire exome region, which enabled more accurate genome wide copy number variation prediction. All mutations detected using the targeted PredicineATLAS panel that were ≥1% allele frequency were also detected by Plasma WES. Furthermore, Plamsa WES identified 1.5-15.3× additional mutations compared with the PredicineATLAS panel.

PredicineECM enzymatic methylation assay was superior to whole genome bisulfite sequencing (WGBS) in reducing DNA damage and GC bias, resulting in increased NGS read mapping rate and quality score. Profiling of 2.5, 5 and 10 ng cfDNA using PredicineECM showed higher mapping rate and quality compared with WGBS profiling of 10 ng cfDNA. Additionally, clustering analysis of CpG methylation using PredicineECM separated tumor samples from normal samples and may serve as additional markers for Minimal residual disease (MRD) tracing or early cancer detection.

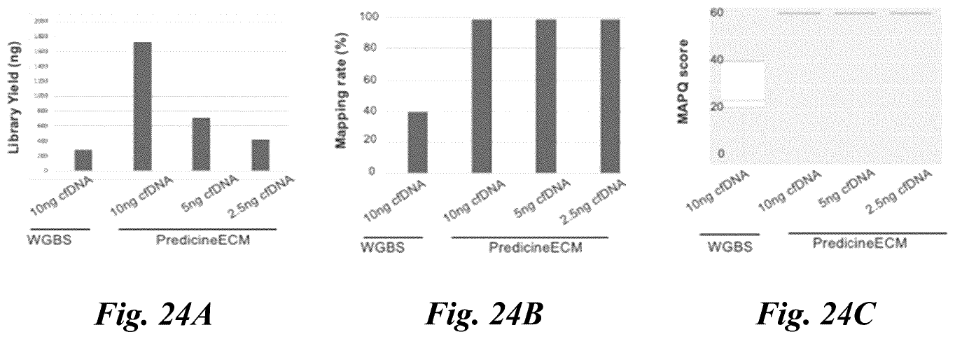

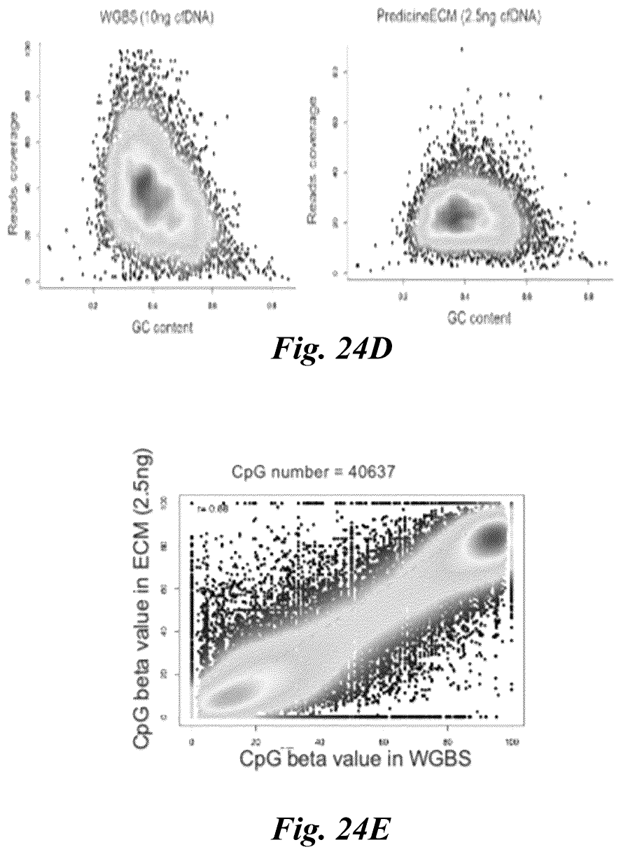

FIGS. 24A-24E show the NGS technical performance comparison of Predicine ECM vs WGBS. FIG. 24A shows the comparisons for library yield, FIG. 24B shows mapping rate, FIG. 24C Mapping quality, FIG. 24D shows Coverage, and FIG. 24E shows correlation plots between PredicineECM WGBS.

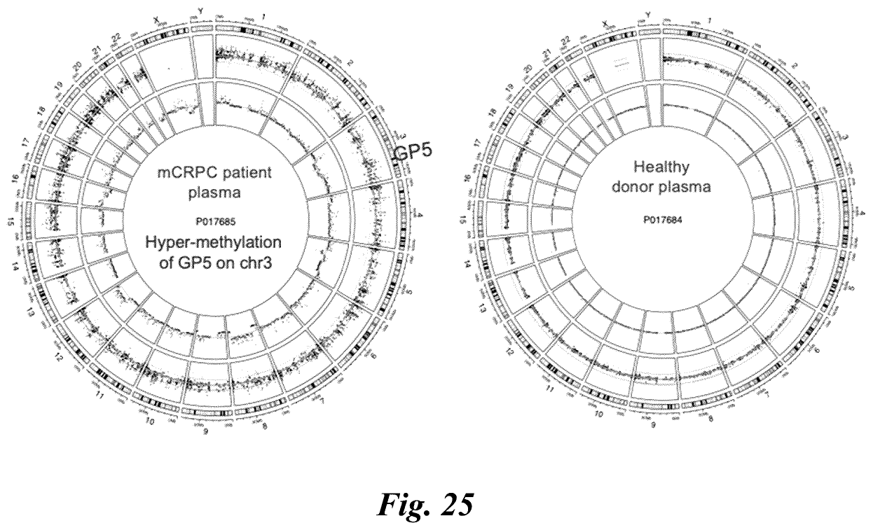

FIG. 25 shows the Differentially methylated region (DMR) analysis of cancer and normal sample, specifically Circos plot of CpG islands DNA methylation signal. The outer circle is the DMRs. Red and Green color represent hyper and hypo methylated DMRs, respectively. Global hypermethylation is observed for the mCRPC patient plasma sample. Inner circle is the WGS CNV results based on methylation data.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-88. (canceled)

89. A method for detecting a presence or an absence of cancer in a subject, comprising:
(a) assaying cell-free deoxyribonucleic acid (cfDNA) molecules and cell-free ribonucleic (cfRNA) molecules from a biological sample obtained or derived from said subject to detect a first set of biomarkers from said cfDNA molecules and a second set of biomarkers from said cfRNA molecules; and
(b) computer processing said first set of biomarkers and said second set of biomarkers to detect said presence or said absence of said cancer in said subject.

90. The method of claim 89, wherein said biological sample is selected from the group consisting of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a saliva sample, tissue biopsy, pleural fluid sample, peritoneal fluid sample, amniotic fluid sample, cerebrospinal fluid sample, lymphatic fluid sample, sweat sample, tear sample, semen sample, or any derivative thereof, and any combination thereof.

91. The method of claim 89, wherein said biological sample comprises said plasma sample or said urine sample.

92. The method of claim 89, wherein (a) comprises subjecting said biological sample to conditions that are sufficient to isolate, enrich, or extract said cfDNA molecules and said set of cfRNA molecules.

93. The method of claim 89, wherein at least one of said cfDNA molecules and said cfRNA molecules are assayed using nucleic acid sequencing to produce nucleic acid sequencing reads.

94. The method of claim 93, further comprising performing at least one of single-stranded consensus calling and double-stranded consensus calling on said nucleic acid sequencing reads, thereby suppressing sequencing and PCR errors in said nucleic acid sequencing reads.

95. The method of claim 89, wherein said cfDNA molecules are assayed using DNA sequencing.

96. The method of claim 95, wherein said DNA sequencing is selected from the group consisting of: next-generation sequencing, whole genome sequencing, low-pass sequencing, targeted sequencing, whole exome sequencing, methylation-aware sequencing, bisulfite sequencing, enzymatic methylation sequencing, and a combination thereof.

97. The method of claim 95, wherein said DNA sequencing comprises low-pass whole genome sequencing or whole exome sequencing.

98. The method of claim 89, wherein said cfRNA molecules are assayed using RNA sequencing.

99. The method of claim 98, wherein said RNA sequencing is selected from the group consisting of: next-generation sequencing, transcriptome sequencing, mRNA-seq, totalRNA-seq, smallRNA-seq, exosome sequencing, and a combination thereof.

100. The method of claim 89, wherein at least one of said cfDNA molecules and said cfRNA molecules are assayed using a polymerase chain reaction (PCR) assay, microarray, or a isothermal amplification.

101. The method of claim 89, wherein said cancer is selected from the group consisting of: breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof.

102. The method of claim 101, wherein said cancer comprises said prostate cancer.

103. The method of claim 101, wherein said cancer comprises said breast cancer

104. The method of claim 101, wherein said cancer comprises said bladder cancer.

105. The method of claim 89, wherein (b) comprises processing said first set of biomarkers and said second set of biomarkers using a trained machine learning algorithm.

106. The method of claim 105, wherein said trained machine learning algorithm comprises a deep learning algorithm, a support vector machine (SVM), a neural network, or a Random Forest.

107. The method of claim 89, further comprising administering a clinical intervention to said subject based at least in part on said detecting in (b), wherein said clinical intervention is selected from the group consisting of: surgical resection, chemotherapy, radiotherapy, immunotherapy, adjuvant therapy, neoadjuvant therapy, androgen deprivation therapy, and a combination thereof.

108. The method of claim 89, wherein said first set of biomarkers comprises quantitative measures of a first set of cancer-associated genomic loci, wherein said first set of cancer-associated genomic loci comprises one or more members selected from the group consisting of genes listed in Table 1, PTEN, TP53, RB1, FGFR3, or ERBB2.

109. The method of claim 89, wherein said second set of biomarkers comprises quantitative measures of a second set of cancer-associated genomic loci, wherein said second set of cancer-associated genomic loci comprises one or more members selected from the group consisting of genes listed in Table 2.

110. The method of claim 89, further comprising using probes configured to selectively enrich said biological sample for nucleic acid molecules corresponding to a set of genomic loci.

111. The method of claim 89, further comprising assaying germline DNA (gDNA) molecules obtained or derived from said subject to detect a third set of biomarkers, and computer processing said third set of biomarkers to detect said presence or said absence of said cancer in said subject.

112. The method of claim 89, wherein said first set of biomarkers from said cfDNA molecules comprise tumor-associated alterations selected from the group consisting of: copy number alterations (CNAs), copy number losses (CNLs), single nucleotide variants (SNVs), insertions or deletions (indels), and rearrangements.

113. The method of claim 89, wherein said second set of biomarkers from said cfRNA molecules comprise tumor-associated alterations selected from the group consisting of: alternative splicing variants, fusions, single nucleotide variants (SNVs), and insertions or deletions (indels).

114. The method of claim 89, further comprising determining a blood copy number burden based on copy number alterations or copy number losses of said first set of biomarkers.

115. The method of claim 89, further comprising determining, among said first set of biomarkers, a mutant allele frequency of a set of somatic mutations.

116. The method of claim 115, further comprising determining a circulating tumor DNA (ctDNA) fraction of said cancer of said subject based at least in part on said set of mutant allele frequencies.

117. The method of claim 115, further comprising determining a plasma tumor mutational burden (pTMB) of said cancer of said subject based at least in part on said set of mutant allele frequencies.

118. A method for detecting a presence or an absence of prostate cancer in a subject, comprising:
(a) assaying cell-free deoxyribonucleic acid (cfDNA) molecules and germline DNA (gDNA) molecules from a biological sample obtained or derived from said subject to detect a first set of biomarkers from said cfDNA molecules and a second set of biomarkers from said gRNA molecules, wherein at least one of said first set of biomarkers and said second set of biomarkers comprises an androgen receptor (AR) alteration; and
(b) computer processing said first set of biomarkers and said second set of biomarkers to detect said presence or said absence of said prostate cancer in said subject.
